(19) United States
(12) Patent Application Publication
(10) Pub. No.: US 2021/0017550 A1
(43) Pub. Date: Jan. 21, 2021

(54) METHOD FOR N-BUTANOL PRODUCTION USING HETEROLOGOUS EXPRESSION OF ANAEROBIC PATHWAYS

(71) Applicants: SILICOLIFE LDA., Braga (PT); UNIVERSIDADE DO MINHO, Braga (PT)

(72) Inventors: Isabel Cristina DE ALMEIDA PEREIRA DA ROCHA, Braga (PT); Miguel Francisco DE ALMEIDA PEREIRA DA ROCHA, Braga (PT); Ana Sofia ARAÚJO FERREIRA, Sequeira, Braga (PT); Filipe Alexandre Wang LIU, Chicago, IL (US); Rui Miguel Pinheiro DA SILVA PEREIRA, Guimarães (PT); Paulo Ricardo CARVALHO VILAÇA, Braga (PT); Simão Pedro DE PINHO SOARES, Braga (PT)

(73) Assignees: SILICOLIFE LDA., Braga (PT); UNIVERSIDADE DO MINHO, Braga (PT)

(21) Appl. No.: 17/042,124

(22) PCT Filed: Mar. 28, 2019
(86) PCT No.: PCT/EP2019/057944

§ 371 Date: Sep. 27, 2020

(30) Foreign Application Priority Data
Mar. 28, 2018 (EP) 18164811.4

(51) Int. Cl. C12P 7/16; C12N 9/04; C12N 9/10; C12N 9/88; C12N 9/02

(52) U.S. Cl. CPC C12P 7/16; C12N 9/0006; C12N 9/13; C12N 9/88; C12N 9/001; C12N 9/0008; C12R 1/01; C12Y 208/03012; C12Y 103/08006; C12Y 101/01099; C12Y 103/01044; C12Y 101/01011; C12Y 102/01003; C12Y 402/01

(57) ABSTRACT

The present invention relates to a method for the production of n-butanol using a transgenic cell with heterologous expression of 2-hydroxyglutarate dehydrogenase, glutaconate-CoA transferase, (R)-2-hydroxyglutaryl-CoA dehydrogenase, glutaryl CoA dehydrogenase, trans-2-enoyl-CoA reductase (NAD+) and bifunctional aldehyde/alcohol dehydrogenase (NAD+).

The present invention relates to a method for the production of n-butanol using a transgenic cell capable of heterologous expression of 2-hydroxyglutarate dehydrogenase, glutaconate-CoA transferase, (R)-2-hydroxyglutaryl-CoA dehydrogenase, glutaryl-CoA dehydrogenase, trans-2-enoyl-CoA reductase (NAD+) and bifunctional aldehyde/alcohol dehydrogenase (NAD+).

### DESCRIPTION

### Background of the Invention

n-butanol occurs naturally as a minor product of the fermentation of sugars and other carbohydrates and is present in many foods and beverages. It is also a permitted artificial food flavouring in the United States. n-butanol can be used as a drop-in chemical key raw material in the production of cleansing agents, paints, coatings, plasticizers and adhesives; it also acts as the precursor in manufacturing acetates, acrylate, glycol ethers and solvents. It is further used as a drop-in chemical replacement of petroleum-based n-butanol in almost all applications. Its use as an additive has resulted in increasing need from the pharmaceutical industry. It is also regarded as a potential bio-fuel with improved properties when compared with bio-ethanol.

Until the mid-1940's, n-butanol was produced predominantly through fermentation using a process called Acetone-Butanol-Ethanol (ABE) fermentation with clostridia bacteria. Recent advances in the fields of biotechnology and bioprocessing have resulted in a renewed interest in the fermentation production of chemicals and fuels, including n-butanol. With continuous fermentation technology, n-butanol can be produced at higher yields, concentrations and production rates. Advanced technology in metabolic engineering and synthetic biology has also improved the development of heterologous metabolic pathways in well-characterized microbial hosts for n-butanol fermentation. The rapidly expanding genomic information, molecular biology techniques, and high-throughput tools resulted in a significant progress in constructing non-native organisms for the production of fuel-grade compounds beyond the scope of what native organisms can produce. The n-butanol process still has to overcome some important milestones, which include: more microorganisms and pathways able to overproduce n-butanol, microbial tolerance to n-butanol concentrations, improved yields, and specificity of n-butanol production vs. co-products such as acetone, increased productivity and finally the use of flexible feedstocks.

The objective of this invention is to provide means and methods that allow for improved n-butanol production.

This objective is attained by the subject-matter of the independent claims of the present specification.

### SUMMARY OF THE INVENTION

A first aspect of the invention relates to a method for production of n-butanol, wherein a transgenic cell heterologously expresses each of the following enzymes:
a. 2-hydroxyglutarate dehydrogenase hgdH (EC 1.1.99.2.);
b. glutaconate-CoA transferase gctAB (EC 2.8.3.12);
c. (R)-2-hydroxyglutaryl-CoA dehydratase subunits A, B and C hgdABC (EC 4.2.1.167);
d. glutaryl CoA dehydrogenase gcdH (EC 1.3.8.6.);
e. trans-2-enoyl-CoA reductase (NAD+) ter (EC 1.3.1.44.) and
f. a bifunctional aldehyde/alcohol dehydrogenase (NAD+) selected from adhE1 and adhE2 (EC 1.1.1.11/1.2.1.3.);

and is grown in a medium comprising a metabolic precursor of 2-oxoglutarate.

A second aspect of the invention relates to a cell heterologously expressing each of the above-mentioned enzymes.

Another aspect of the invention relates to a plurality of plasmids comprising genes encoding the above-mentioned enzymes.

Certain aspects of the invention may be summarized as a novel and unexpectedly advantageous combination of a first set of three reactions capable of producing glutaconate, namely 2-hydroxyglutarate dehydrogenase hgdH, glutaconate-CoA transferase gctAB, and (R)-2-hydroxyglutaryl-CoA dehydratase hgdABC, with the last three steps common to the clostridial pathway (butanol's native producers) through an enzyme never used before to the production of butanol, namely glutaryl-CoA dehydrogenase (encoded by the gene gcdH).

### Terms and Definitions

The term hgdH in the context of the present specification relates to 2-hydroxyglutarate dehydrogenase, EC 1.1.99.2.

The term gctAB in the context of the present specification relates to glutaconate-CoA transferase subunits A and B, EC 2.8.3.12.

The term hgdABC in the context of the present specification relates to (R)-2-hydroxyglutaryl-CoA dehydrogenase subunits A, B and C, EC 4.2.1.167.

The term gcdH in the context of the present specification relates to glutaryl-CoA dehydrogenase, EC 1.3.8.6.

The term ter in the context of the present specification relates to trans-2-enoyl-CoA reductase (NAD+), EC 1.3.1.44.

The terms adhE, adhE1 or adhE2 in the context of the present specification relate to bifunctional aldehyde/alcohol dehydrogenase (NAD+), EC 1.1.1.11/1.2.1.3.

The term bp in the context of the present specification is an abbreviation for base pairs, while kbp is an abbreviation for kilo base pairs.

Amino acid sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids.

In the context of the present specifications the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

In the context of the present specification, the terms anaerobic or aerobic refer to a culture or growth condition, wherein the amount of dissolved oxygen is null in the case of anaerobic conditions and >10% of saturation for aerobic conditions. An anaerobic bacterium does not require oxygen for growth. Strictly anaerobic bacteria require oxygen-free conditions for survival, while facultatively anaerobic bacteria can grow under either oxygen-enriched or oxygen-free conditions.

In the context of the present specification, the term heterologous refers to a gene or protein derived from a source other than the host species whereas homologous refers to a gene or protein derived from the host microbial organism.

In the context of the present specification, the term plasmid or plasmids refer to a small (1.5 to 15 kb, particularly 2-10 kb), circular piece of double-stranded DNA comprising an origin of replication operable in a host cell, and a selection marker gene.

In the context of the present specification, the term codon-optimized as it refers to genes or coding regions of nucleic acid molecules for transformation of specific hosts, refers to the alteration of codons in the gene or coding regions of the nucleic acid molecules to reflect the typical codon usage of the host organism without altering the polypeptide encoded by the DNA. Each codon is recognized by a transfer RNA (tRNA) that translates the codon to an amino acid. There are bioinformatic methods available that search for the most prevalent tRNAs of a host organism for each codon and optimize the codons with respect to the host organism thereby potentially increasing the expression rate.

### DETAILED DESCRIPTION OF THE INVENTION

In a bioinformatics approach, possible heterologous pathways to produce n-butanol in a transgenic bacterial cell were proposed using enumeration methodologies based on (Liu, F. et al. (2015) Computer Methods and Programs in Biomedicine, 118(2), pp. 134-146). The solutions obtained were analyzed computationally using a proprietary digital platform from SilicoLife. The ranking and evaluation process involved diverse criteria such as novelty, size of pathway, n-butanol yield, conservation of number of carbon atoms. OptFlux (Rocha, I. et al. (2010), BMC Systems Biology, 4(1), p. 45) and a proprietary digital platform from SilicoLife were used to perform all simulations. Flux Balance Analysis (FBA) and variants were used as simulation methods. The most promising pathway was translated into laboratory experiments and optimized.

A first aspect of the invention relates to a method for production of n-butanol, wherein a transgenic cell heterologously or endogenously, particularly heterologously, expresses each of the following enzymes:
a. 2-hydroxyglutarate dehydrogenase hgdH (EC 1.1.99.2.);
b. glutaconate-CoA transferase gctAB (EC 2.8.3.12);
c. (R)-2-hydroxyglutaryl-CoA dehydratase subunits A, B and C hgdABC (EC 4.2.1.167);
d. glutaryl CoA dehydrogenase gcdH (EC 1.3.8.6.);
e. trans-2-enoyl-CoA reductase (NAD+) ter (EC 1.3.1.44.) and
f. a bifunctional aldehyde/alcohol dehydrogenase (NAD+) selected from adhE1 and adhE2 (EC 1.1.1.11/1.2.1.3.);

and is grown in a medium comprising a metabolic precursor of 2-oxoglutarate.

In certain embodiments, n-butanol is extracted from said medium. In certain embodiments, n-butanol is extracted from said medium via distillation.

In certain embodiments, said metabolic precursor of 2-oxoglutarate is selected from glucose, glycerol, glutamate or acetate.

In certain embodiments, the transgenic cell is a bacterium or a yeast cell.

In certain embodiments, the bacterium or the yeast cell is selected from genera Escherichia, Corynebacterium, Ralstonia, Clostridium, Pseudomonas, Lactobacillus, Lactococcus, Acidaminococcus, Fusobacterium, Peptoniphilus, Saccharomyces, Streptomyces Lactobacillus, Pichia, Kluyveromyces, Yarrowia, or Staphylococci, particularly Escherichia coli.

In certain embodiments of the method of the invention, the protein hgdH is encoded by a gene derived from a strictly or facultatively anaerobic bacterium. In certain embodiments of the method of the invention, said hgdH is at least 60%, 65%, 70%, 75%, 80%, 85%, 90%, 95% or >95% identical, with respect to its amino acid sequence, to hgdH of Acidaminococcus fermentans. In certain embodiments of the method of the invention, hgdH is at least 60%, 65%, 70%, 75%, 80%, 85%, 90%, 95% or >95% identical to SEQ NO 1 and has a catalytic activity of at least 75% of the activity of SEQ NO 1.

In certain embodiments of the method of the invention, the protein gctAB is encoded by a gene derived from a strictly or facultatively anaerobic bacterium. In certain embodiments of the method of the invention, said gctAB is at least 60%, 65%, 70%, 75%, 80%, 85%, 90%, 95% or >95% identical, with respect to its amino acid sequence, to gctAB of Acidaminococcus fermentans. In certain embodiments of the method of the invention, subunit A of gctAB is at least 60%, 65%, 70%, 75%, 80%, 85%, 90%, 95% or >95% identical to SEQ NO 2 and has a catalytic activity of at least 75% of the activity of SEQ NO 2. In certain embodiments of the method of the invention, subunit B of gctAB is at least 60%, 65%, 70%, 75%, 80%, 85%, 90%, 95% or >95% identical to SEQ NO 3 and has a catalytic activity of at least 75% of the activity of SEQ NO 3.

In certain embodiments of the method of the invention, the A subunit of the protein hgd is encoded by a gene derived from a strictly or facultatively anaerobic bacterium. In certain embodiments of the method of the invention, said hgdA is at least 60%, 65%, 70%, 75%, 80%, 85%, 90%, 95% or >95% identical, with respect to its amino acid sequence, to hgdA of Clostridium symbiosum. In certain embodiments of the method of the invention, hgdA is at least 60%, 65%, 70%, 75%, 80%, 85%, 90%, 95% or >95% identical to SEQ NO 4 and has a catalytic activity of at least 75% of the activity of SEQ NO 4.

In certain embodiments of the method of the invention, the B subunit of the protein hgd is encoded by a gene derived from a strictly or facultatively anaerobic bacterium. In certain embodiments of the method of the invention, said hgdB is at least 60%, 65%, 70%, 75%, 80%, 85%, 90%, 95% or >95% identical, with respect to its amino acid sequence, to hgdB of Clostridium symbiosum. In certain embodiments of the method of the invention, hgdB is at least 60%, 65%, 70%, 75%, 80%, 85%, 90%, 95% or >95% identical to SEQ NO 5 and has a catalytic activity of at least 75% of the activity of SEQ NO 5.

In certain embodiments of the method of the invention, the C subunit of the protein hgd is encoded by a gene derived from a strictly or facultatively anaerobic bacterium. In certain embodiments of the method of the invention, said hgdC is at least 60%, 65%, 70%, 75%, 80%, 85%, 90%, 95% or >95% identical, with respect to its amino acid sequence, to hgdC of Acidaminococcus fermentans. In certain embodiments of the method of the invention, hgdC is at least 60%, 65%, 70%, 75%, 80%, 85%, 90%, 95% or >95% identical to SEQ NO 6 and has a catalytic activity of at least 75% of the activity of SEQ NO 6.

In certain embodiments of the method of the invention, the protein gcdH is encoded by a gene derived from a strictly or facultatively anaerobic bacterium. In certain embodiments of the method of the invention, said gcdH is at least 60%, 65%, 70%, 75%, 80%, 85%, 90%, 95% or >95% identical, with respect to its amino acid sequence, to gcdH of Pseudomonas aeruginosa. In certain embodiments of the method of the invention, gcdH is at least 60%, 65%, 70%, 75%, 80%, 85%, 90%, 95% or >95% identical to SEQ NO 7 and has a catalytic activity of at least 75% of the activity of SEQ NO 7.

In certain embodiments of the method of the invention, the protein ter is encoded by a gene derived from a strictly or facultatively anaerobic bacterium. In certain embodiments of the method of the invention, said ter is at least 60%, 65%, 70%, 75%, 80%, 85%, 90%, 95% or >95% identical, with respect to its amino acid sequence, to ter of Treponema denticola. In certain embodiments of the method of the invention, ter is at least 60%, 65%, 70%, 75%, 80%, 85%, 90%, 95% or >95% identical to SEQ NO 8 and has a catalytic activity of at least 75% of the activity of SEQ NO 8.

In certain embodiments of the method of the invention, the protein adhE1 is encoded by a gene derived from a strictly or facultatively anaerobic bacterium. In certain embodiments of the method of the invention, said adhE1 is at least 60%, 65%, 70%, 75%, 80%, 85%, 90%, 95% or >95% identical, with respect to its amino acid sequence, to adhE1 of Clostridium acetobutylicum. In certain embodiments of the method of the invention, adhE1 is at least 60%, 65%, 70%, 75%, 80%, 85%, 90%, 95% or >95% identical to SEQ NO 9 and has a catalytic activity of at least 75% of the activity of SEQ NO 9.

In certain embodiments of the method of the invention, the protein adhE2 is encoded by a gene derived from a strictly or facultatively anaerobic bacterium. In certain embodiments of the method of the invention, said adhE2 is at least 60%, 65%, 70%, 75%, 80%, 85%, 90%, 95% or >95% identical, with respect to its amino acid sequence, to adhE2 of Clostridium acetobutylicum. In certain embodiments of the method of the invention, adhE2 is at least 60%, 65%, 70%, 75%, 80%, 85%, 90%, 95% or >95% identical to SEQ NO 13 and has a catalytic activity of at least 75% of the activity of SEQ NO 13.

In certain embodiments, said transgenic cell comprises one or more plasmids encoding said heterologously expressed enzymes under control of a promoter sequence operable in said cell, particularly a T7 promoter, a lac promoter, a trp promoter, a tac promoter or a APL promoter.

In certain embodiments, said fermentation step is performed under anaerobic conditions at 25 to 37° C., particularly at 30° C.

In certain embodiments, the medium comprises 8-12 g·L⁻¹ glucose, 8-10 g·L⁻¹ dibasic sodium phosphate dihydrate, 6-8 g·L⁻¹ monobasic potassium phosphate, 0.5-0.7 g·L⁻¹ sodium chloride, 1.2-1.5 g·L⁻¹ magnesium sulphate, 0.03-0.05 g·L⁻¹ calcium chloride dihydrate, 0.8-1.2 g·L⁻¹ ammonium chloride, and 8-12 mmol·L⁻¹ sodium bicarbonate, 0.1-0.15 μg·L⁻¹ selenium, 0.08-0.12 μg·L⁻¹ nickel, 0.7-0.9 μg·L⁻¹ molybdenum, ampicillin, spectinomycin, and kanamycin and neutral pH, particularly pH 6.8-7.3.

In certain embodiments, said plasmid comprises a lac, tac or T7 promoter, and the expression of said heterologous genes is induced by adding Isopropyl β-D-1-thiogalactopyranosid (IPTG) to the medium, particularly 0.1-1 mmol·L⁻¹ IPTG, more particularly 0.5 mmol·L⁻¹ IPTG. In certain embodiments, a T7-RNA-polymerase is under control of a lac promoter and when IPTG is added, the T7-RNA-polymerase is expressed and transcribes the protein under control of a T7 promoter.

In certain embodiments, said plasmid comprises a trp promoter, and the expression of heterologous genes is induced by adding 3-b-indoleacrylic acid to the medium, at concentrations ranging from 10 μg·mL⁻¹ to 100 μg·mL⁻¹.

In certain embodiments, said plasmid comprises a APL promoter, and the expression of heterologous genes is induced by increasing the temperature to 42° C.

A second aspect of the invention relates to a transgenic cell, wherein each of the following enzymes are expressed:
a. 2-hydroxyglutarate dehydrogenase hgdH (EC 1.1.99.2.);
b. glutaconate-CoA transferase gctAB (EC 2.8.3.12);
c. (R)-2-hydroxyglutaryl-CoA dehydratase subunits A, B and C hgdABC (EC 4.2.1.167);
d. glutaryl CoA dehydrogenase gcdH (EC 1.3.8.6.);
e. trans-2-enoyl-CoA reductase (NAD+) ter (EC 1.3.1.44.); and
f. a bifunctional aldehyde/alcohol dehydrogenase (NAD+) selected from adhE1 and adhE2 (EC 1.1.1.11/1.2.1.3.).

In certain embodiments of the transgenic cell of the invention, at least 4 of said enzymes are expressed heterologously. In certain embodiments of the transgenic cell, 5 or 6 enzymes are expressed heterologously.

In certain embodiments, the cell is selected from genera Escherichia, Corynebacterium, Ralstonia, Clostridium, Pseudomonas, Lactobacillus, Lactococcus, Acidaminococcus, Fusobacterium, Peptoniphilus, Saccharomyces, Streptomyces Lactobacillus, Pichia, Kluyveromyces, Yarrowia, or Staphylococci, particularly Escherichia coli.

In certain embodiments of the transgenic cell of the invention, the protein hgdH is encoded by a gene derived from a strictly or facultatively anaerobic bacterium. In certain embodiments of the transgenic cell of the invention, said hgdH is at least 60%, 65%, 70%, 75%, 80%, 85%, 90%, 95% or >95% identical, with respect to its amino acid sequence, to hgdH of Acidaminococcus fermentans. In certain embodiments of the transgenic cell of the invention, hgdH is at least 60%, 65%, 70%, 75%, 80%, 85%, 90%, 95% or >95% identical to SEQ NO: 1 and has a catalytic activity of at least 75% of the activity of SEQ NO 1.

In certain embodiments of the transgenic cell of the invention, the protein gctAB is encoded by a gene derived from a strictly or facultatively anaerobic bacterium. In certain embodiments of the transgenic cell of the invention, said gctAB is at least 60%, 65%, 70%, 75%, 80%, 85%, 90%, 95% or >95% identical, with respect to its amino acid sequence, to gctAB of Acidaminococcus fermentans. In certain embodiments of the transgenic cell of the invention, subunit A of gctAB is at least 60%, 65%, 70%, 75%, 80%, 85%, 90%, 95% or >95% identical to SEQ NO 2 and has a catalytic activity of at least 75% of the activity of SEQ NO 2. In certain embodiments of the transgenic cell of the invention, subunit B of gctAB is at least 60%, 65%, 70%, 75%, 80%, 85%, 90%, 95% or >95% identical to SEQ NO 3 and has a catalytic activity of at least 75% of the activity of SEQ NO 3.

In certain embodiments of the transgenic cell of the invention, the A subunit of the protein hgd is encoded by a gene derived from a strictly or facultatively anaerobic bacterium. In certain embodiments of the transgenic cell of the invention, said hgdA is at least 60%, 65%, 70%, 75%, 80%, 85%, 90%, 95% or >95% identical, with respect to its amino acid sequence, to hgdA of Clostridium symbiosum. In certain embodiments of the transgenic cell of the invention, hgdA is at least 60%, 65%, 70%, 75%, 80%, 85%, 90%, 95% or >95% identical to SEQ NO 4 and has a catalytic activity of at least 75% of the activity of SEQ NO 4.

In certain embodiments of the transgenic cell of the invention, the B subunit of the protein hgd is encoded by a gene derived from a strictly or facultatively anaerobic bacterium. In certain embodiments of the transgenic cell of the invention, said hgdB is at least 60%, 65%, 70%, 75%, 80%, 85%, 90%, 95% or >95% identical, with respect to its amino acid sequence, to hgdB of Clostridium symbiosum. In certain embodiments of the transgenic cell of the invention, hgdB is at least 60%, 65%, 70%, 75%, 80%, 85%, 90%, 95% or >95% identical to SEQ NO 5 and has a catalytic activity of at least 75% of the activity of SEQ NO 5.

In certain embodiments of the transgenic cell of the invention, the C subunit of the protein hgd is encoded by a gene derived from a strictly or facultatively anaerobic bacterium. In certain embodiments of the transgenic cell of the invention, said hgdC is at least 60%, 65%, 70%, 75%, 80%, 85%, 90%, 95% or >95% identical, with respect to its amino acid sequence, to hgdC of Acidaminococcus fermentans. In certain embodiments of the transgenic cell of the invention, hgdC is at least 60%, 65%, 70%, 75%, 80%, 85%, 90%, 95% or >95% identical to SEQ NO 6 and has a catalytic activity of at least 75% of the activity of SEQ NO 6.

In certain embodiments of the transgenic cell of the invention, the protein gcdH is encoded by a gene derived from a strictly or facultatively anaerobic bacterium. In certain embodiments of the transgenic cell of the invention, said gcdH is at least 60%, 65%, 70%, 75%, 80%, 85%, 90%, 95% or >95% identical, with respect to its amino acid sequence, to gcdH of Pseudomonas aeruginosa. In certain embodiments of the transgenic cell of the invention, gcdH is at least 60%, 65%, 70%, 75%, 80%, 85%, 90%, 95% or >95% identical to SEQ NO 7 and has a catalytic activity of at least 75% of the activity of SEQ NO 7.

In certain embodiments of the transgenic cell of the invention, the protein ter is encoded by a gene derived from a strictly or facultatively anaerobic bacterium. In certain embodiments of the transgenic cell of the invention, said ter is at least 60%, 65%, 70%, 75%, 80%, 85%, 90%, 95% or >95% identical, with respect to its amino acid sequence, to ter of Treponema denticola. In certain embodiments of the transgenic cell of the invention, ter is at least 60%, 65%, 70%, 75%, 80%, 85%, 90%, 95% or >95% identical to SEQ NO 8 and has a catalytic activity of at least 75% of the activity of SEQ NO 8.

In certain embodiments of the transgenic cell of the invention, the protein adhE1 is encoded by a gene derived from a strictly or facultatively anaerobic bacterium. In certain embodiments of the transgenic cell of the invention, said adhE1 is at least 60%, 65%, 70%, 75%, 80%, 85%, 90%, 95% or >95% identical, with respect to its amino acid sequence, to adhE1 of Clostridium acetobutylicum. In certain embodiments of the transgenic cell of the invention, adhE1 is at least 60%, 65%, 70%, 75%, 80%, 85%, 90%, 95% or >95% identical to SEQ NO 9 and has a catalytic activity of at least 75% of the activity of SEQ NO 9.

In certain embodiments of the transgenic cell of the invention, the protein adhE2 is encoded by a gene derived from a strictly or facultatively anaerobic bacterium. In certain embodiments of the transgenic cell of the invention, said adhE2 is at least 60%, 65%, 70%, 75%, 80%, 85%, 90%, 95% or >95% identical, with respect to its amino acid sequence, to adhE2 of Clostridium acetobutylicum. In certain embodiments of the transgenic cell of the invention, adhE2 is at least 60%, 65%, 70%, 75%, 80%, 85%, 90%, 95% or >95% identical to SEQ NO 13 and has a catalytic activity of at least 75% of the activity of SEQ NO 13.

In certain embodiments, said cell comprises the sequences for said heterologously expressed enzymes under control of a promoter sequence operable in said cell. In certain embodiments, the promoter is a T7 promoter, a lac promoter, a trp promoter, a tac promoter or a APL promoter.

A third aspect of the invention relates to a medium for n-butanol production comprising 8-12 g·L⁻¹ glucose, 8-10 g·L⁻¹ dibasic sodium phosphate dihydrate, 6-8 g·L⁻¹ monobasic potassium phosphate, 0.5-0.7 g·L⁻¹ sodium chloride, 1.2-1.5 g·L⁻¹ magnesium sulphate, 0.03-0.05 g·L⁻¹ calcium chloride dihydrate, 0.8-1.2 g·L⁻¹ ammonium chloride, and 8-12 mmol·L⁻¹ sodium bicarbonate, 0.1-0.15 μg·L⁻¹ selenium, 0.08-0.12 μg·L⁻¹ nickel, 0.7-0.9 μg·L⁻¹ molybdenum, ampicillin, spectinomycin, and kanamycin and neutral pH, particularly pH 6.8-7.3.

A fourth aspect of the invention relates to a plurality of plasmids comprising genes encoding
a. 2-hydroxyglutarate dehydrogenase hgdH (EC 1.1.99.2.);
b. glutaconate-CoA transferase gctAB (EC 2.8.3.12);
c. (R)-2-hydroxyglutaryl-CoA dehydratase subunits A, B and C hgdABC (EC 4.2.1.167);
d. glutaryl CoA dehydrogenase gcdH (EC 1.3.8.6.);
e. trans-2-enoyl-CoA reductase (NAD+) ter (EC 1.3.1.44.) and
f. a bifunctional aldehyde/alcohol dehydrogenase (NAD+) selected from adhE1 and adhE2 (EC 1.1.1.11/1.2.1.3.).

In certain embodiments, each plasmid in said plurality of plasmids comprises more than one of said genes and each of said plasmids comprises a different selection marker. In certain embodiments, the plurality of plasmids consists of three plasmids, each encoding two of said genes.

In certain embodiments, each plasmid independently of each other comprises a promoter sequence operable in a desired target cell, particularly a T7 promoter, a lac promoter, a trp promoter, a tac promoter or a λP_{L} promoter.

In certain embodiments, one plasmid comprises the genes encoding gctAB and hgdH and a gene for spectinomycin resistance and having the size of about 6.5 kbp, wherein particularly the one plasmid further comprises a T7 promoter sequence. In certain embodiments, one plasmid has the sequence SEQ NO 10.

In certain embodiments, one plasmid comprises the genes encoding hgdABC and gcdH and a gene for kanamycin resistance and having the size of about 8.3 kbp, wherein particularly the one plasmid further comprises a T7 promoter sequence. In certain embodiments, one plasmid has the sequence SEQ NO 11.

In certain embodiments, one plasmid comprises the genes encoding adhE1 or adhE2 and ter and a gene for ampicillin resistance and having the size of about 9.1 kbp, wherein particularly the one plasmid further comprises a T7 promoter sequence. In certain embodiments, one plasmid has the sequence SEQ NO 12.

A fifth aspect of the invention relates to a kit or set of parts comprising the said transgenic cell or said plasmids and said medium.

Wherever alternatives for single separable features such as, for example, an isotype protein or coding sequence, an organism genus or a concentration of a chemical are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

Items
1. A method for production of n-butanol, wherein a transgenic cell heterologously or endogenously, particularly heterologously, expressing each of the following enzymes:
   a. 2-hydroxyglutarate dehydrogenase hgdH (EC 1.1.99.2.);
   b. glutaconate-CoA transferase gctAB (EC 2.8.3.12);
   c. (R)-2-hydroxyglutaryl-CoA dehydratase subunits A, B and C hgdABC (EC 4.2.1.167);
   d. glutaryl CoA dehydrogenase gcdH (EC 1.3.8.6.);
   e. trans-2-enoyl-CoA reductase (NAD+) ter (EC 1.3.1.44.); and
   f. a bifunctional aldehyde/alcohol dehydrogenase (NAD+) selected from adhE1 and adhE2 (EC 1.1.1.11/1.2.1.3.);
   is grown in a medium comprising a metabolic precursor of 2-oxoglutarate.
2. The method according to item 1, wherein n-butanol is extracted from said medium.
3. The method according to item 1 or 2, wherein said metabolic precursor of 2-oxoglutarate is selected from glucose, glycerol, glutamate or acetate.
4. The method according to any one of items 1 to 3, wherein the transgenic cell is a bacterium or a yeast cell.
5. The method according to item 4, wherein the bacterium or the yeast cell is selected from genera Escherichia, Corynebacterium, Ralstonia, Clostridium, Pseudomonas, Lactobacillus, Lactococcus, Acidaminococcus, Fusobacterium, Peptoniphilus, Saccharomyces, Streptomyces Lactobacillus, Pichia, Kluyveromyces, Yarrowia, or Staphylococci, particularly Escherichia coli.
6. The method according to any one of the preceding items, wherein
   a. the protein hgdH is encoded by a gene derived from a strictly or facultatively anaerobic bacterium, particularly said hgdH is at least 60%, 65%, 70%, 75%, 80%, 85%, 90%, 95% or >95% identical, with respect to its amino acid sequence, to hgdH of Acidaminococcus fermentans, more particularly hgdH is at least 60%, 65%, 70%, 75%, 80%, 85%, 90%, 95% or >95% identical to SEQ NO 1 and has a catalytic activity of at least 75% of the activity of SEQ NO 1 and/or
   b. the protein gctAB is encoded by a gene derived from a strictly or facultatively anaerobic bacterium, particularly said gctAB is at least 60%, 65%, 70%, 75%, 80%, 85%, 90%, 95% or >95% identical, with respect to its amino acid sequence, to gctAB of Acidaminococcus fermentans, more particularly subunit A of gctAB is at least 60%, 65%, 70%, 75%, 80%, 85%, 90%, 95% or >95% identical to SEQ NO 2 and has a catalytic activity of at least 75% of the activity of SEQ NO 2 and/or subunit B of gctAB is at least 60%, 65%, 70%, 75%, 80%, 85%, 90%, 95% or >95% identical to SEQ NO 3 and has a catalytic activity of at least 75% of the activity of SEQ NO 3 and/or
   c. the A subunit of the protein hgd is encoded by a gene derived from a strictly or facultatively anaerobic bacterium, particularly said hgdA is at least 60%, 65%, 70%, 75%, 80%, 85%, 90%, 95% or >95% identical, with respect to its amino acid sequence, to hgdA of Clostridium symbiosum, more particularly hgdA is at least 60%, 65%, 70%, 75%, 80%, 85%, 90%, 95% or >95% identical to SEQ NO 4 and has a catalytic activity of at least 75% of the activity of SEQ NO 4 and/or
   d. the B subunit of the protein hgd is encoded by a gene derived from a strictly or facultatively anaerobic bacterium, particularly said hgdB is at least 60%, 65%, 70%, 75%, 80%, 85%, 90%, 95% or >95% identical, with respect to its amino acid sequence, to hgdB of Clostridium symbiosum, more particularly hgdB is at least 60%, 65%, 70%, 75%, 80%, 85%, 90%, 95% or >95% identical to SEQ NO 5 and has a catalytic activity of at least 75% of the activity of SEQ NO 5 and/or
   e. the C subunit of the protein hgd is encoded by a gene derived from a strictly or facultatively anaerobic bacterium, particularly said hgdC is at least 60%, 65%, 70%, 75%, 80%, 85%, 90%, 95% or >95% identical, with respect to its amino acid sequence, to hgdC of Acidaminococcus fermentans, more particularly hgdC is at least 60%, 65%, 70%, 75%, 80%, 85%, 90%, 95% or >95% identical to SEQ NO 6 and has a catalytic activity of at least 75% of the activity of SEQ NO 6 and/or
   f. the protein gcdH is encoded by a gene derived from a strictly or facultatively anaerobic bacterium, particularly said gcdH is at least 60%, 65%, 70%, 75%, 80%, 85%, 90%, 95% or >95% identical, with respect to its amino acid sequence, to gcdH of Pseudomonas aeruginosa, more particularly gcdH is at least 60%, 65%, 70%, 75%, 80%, 85%, 90%, 95% or >95% identical to SEQ NO 7 and has a catalytic activity of at least 75% of the activity of SEQ NO 7 and/or
   g. the protein ter is encoded by a gene derived from a strictly or facultatively anaerobic bacterium, particularly said ter is at least 60%, 65%, 70%, 75%, 80%, 85%, 90%, 95% or >95% identical, with respect to its amino acid sequence, to ter of Treponema denticola, more particularly ter is at least 60%, 65%, 70%, 75%, 80%, 85%, 90%, 95% or >95% identical to SEQ NO 8 and has a catalytic activity of at least 75% of the activity of SEQ NO 8 and/or
   h. the protein adhE1 is encoded by a gene derived from a strictly or facultatively anaerobic bacterium, particularly said adhE1 is at least 60%, 65%, 70%, 75%, 80%, 85%, 90%, 95% or >95% identical, with respect to its amino acid sequence, to adhE1 of Clostridium acetobutylicum, more particularly adhE1 is at least 60%, 65%, 70%, 75%, 80%, 85%, 90%, 95% or >95% identical to SEQ NO 9 and has a catalytic activity of at least 75% of the activity of SEQ NO 9 and/or
   i. the protein adhE2 is encoded by a gene derived from a strictly or facultatively anaerobic bacterium, particularly said adhE2 is at least 60%, 65%, 70%, 75%, 80%, 85%, 90%, 95% or >95% identical, with respect to its amino acid sequence, to adhE2 of Clostridium acetobutylicum, more particularly adhE2 is at least 60%, 65%, 70%, 75%, 80%, 85%, 90%, 95% or >95% identical to SEQ NO 13 and has a catalytic activity of at least 75% of the activity of SEQ NO 13.
7. The method according to any one of the preceding items, wherein said transgenic cell comprises one or more plasmids encoding said heterologously expressed enzymes under control of a promoter sequence operable in said cell, particularly a T7 promoter, a lac promoter, a trp promoter, a tac promoter or a λP_{L} promoter.
8. The method according to any one of the preceding items, wherein said fermentation step is performed under anaerobic conditions at 25 to 37° C., particularly at 30° C.
9. The method according to any one of the preceding items, wherein the medium comprises 8-12 g·L⁻¹ glucose, 8-10 g·L⁻¹ dibasic sodium phosphate dihydrate, 6-8 g·L⁻¹ monobasic potassium phosphate, 0.5-0.7 g·L⁻¹ sodium chloride, 1.2-1.5 g·L⁻¹ magnesium sulphate, 0.03-0.05 g·L⁻¹ calcium chloride dihydrate, 0.8-1.2 g·L⁻¹ ammonium chloride, and 8-12 mmol·L⁻¹ sodium bicarbonate, 0.1-0.15 μg·L⁻¹ selenium, 0.08-0.12 μg·L⁻¹ nickel, 0.7-0.9 μg·L⁻¹ molybdenum, ampicillin, spectinomycin, and kanamycin and neutral pH, particularly pH 6.8-7.3.
10. The method according to any one of the preceding items 7 to 9, wherein said plasmid comprises
   a. a lac, tac or T7 promoter, and the expression of said heterologous genes is induced by adding IPTG (Isopropyl β-D-1-thiogalactopyranosid) to the medium, particularly 0.1-1 mmol·L⁻¹ IPTG, more particularly 0.5 mmol·L⁻¹ IPTG;
   b. a trp promoter, and the expression of heterologous genes is induced by adding 3-b-indoleacrylic acid to the medium, at concentrations ranging from 10 μg·mL⁻¹ to 100 μg/m·L⁻¹;
   c. a λP_{L} promoter, and the expression of heterologous genes is induced by increasing the temperature to 42° C.
11. A transgenic cell, wherein the following enzymes are expressed:
   a. 2-hydroxyglutarate dehydrogenase hgdH (EC 1.1.99.2.);
   b. glutaconate-CoA transferase gctAB (EC 2.8.3.12);
   c. (R)-2-hydroxyglutaryl-CoA dehydratase subunits A, B and C hgdABC (EC 4.2.1.167);
   d. glutaryl CoA dehydrogenase gcdH (EC 1.3.8.6.);
   e. trans-2-enoyl-CoA reductase (NAD+) ter (EC 1.3.1.44.); and
   f. a bifunctional aldehyde/alcohol dehydrogenase (NAD+) selected from adhE1 and adhE2 (EC 1.1.1.11/1.2.1.3.);
   wherein at least 4 enzymes are expressed heterologously, particularly 5 or 6 enzymes are expressed heterologously.
12. The cell according to item 11, wherein the cell is selected from genera Escherichia, Corynebacterium, Ralstonia, Clostridium, Pseudomonas, Lactobacillus, Lactococcus, Acidaminococcus, Fusobacterium, Peptoniphilus, Saccharomyces, Streptomyces Lactobacillus, Pichia, Kluyveromyces, Yarrowia, or Staphylococci, particularly Escherichia coli.
13. The cell according to item 11 or 12, wherein
   a. the protein hgdH is encoded by a gene derived from a strictly or facultatively anaerobic bacterium, particularly said hgdH is at least 60%, 65%, 70%, 75%, 80%, 85%, 90%, 95% or >95% identical, with respect to its amino acid sequence, to hgdH of Acidaminococcus fermentans, more particularly hgdH is at least 60%, 65%, 70%, 75%, 80%, 85%, 90%, 95% or >95% identical to SEQ NO 1 and has a catalytic activity of at least 75% of the activity of SEQ NO 1 and/or
   b. the protein gctAB is encoded by a gene derived from a strictly or facultatively anaerobic bacterium, particularly said gctAB is at least 60%, 65%, 70%, 75%, 80%, 85%, 90%, 95% or >95% identical, with respect to its amino acid sequence, to gctAB of Acidaminococcus fermentans, more particularly gctAB is at least 60%, 65%, 70%, 75%, 80%, 85%, 90%, 95% or >95% identical to SEQ NO 2 and has a catalytic activity of at least 75% of the activity of SEQ NO 2 and/or subunit B of gctAB is at least 60%, 65%, 70%, 75%, 80%, 85%, 90%, 95% or >95% identical to SEQ NO 3 and has a catalytic activity of at least 75% of the activity of SEQ NO 3 and/or
   c. the A subunit of the protein hgd is encoded by a gene derived from a strictly or facultatively anaerobic bacterium, particularly said hgdA is at least 60%, 65%, 70%, 75%, 80%, 85%, 90%, 95% or >95% identical, with respect to its amino acid sequence, to hgdA of Clostridium symbiosum, more particularly hgdA is at least 60%, 65%, 70%, 75%, 80%, 85%, 90%, 95% or >95% identical to SEQ NO 4 and has a catalytic activity of at least 75% of the activity of SEQ NO 4 and/or
   d. the B subunit of the protein hgd is encoded by a gene derived from a strictly or facultatively anaerobic bacterium, particularly said hgdB is at least 60%, 65%, 70%, 75%, 80%, 85%, 90%, 95% or >95% identical, with respect to its amino acid sequence, to hgdB of Clostridium symbiosum, more particularly hgdB is at least 60%, 65%, 70%, 75%, 80%, 85%, 90%, 95% or >95% identical to SEQ NO 5 and has a catalytic activity of at least 75% of the activity of SEQ NO 5 and/or
   e. the C subunit of the protein hgd is encoded by a gene derived from a strictly or facultatively anaerobic bacterium, particularly said hgdC is at least 60%, 65%, 70%, 75%, 80%, 85%, 90%, 95% or >95% identical, with respect to its amino acid sequence, to hgdC of Acidaminococcus fermentans, more particularly hgdC is at least 60%, 65%, 70%, 75%, 80%, 85%, 90%, 95% or >95% identical to SEQ NO 6 and has a catalytic activity of at least 75% of the activity of SEQ NO 6 and/or
   f. the protein gcdH is encoded by a gene derived from a strictly or facultatively anaerobic bacterium, particularly said gcdH is at least 60%, 65%, 70%, 75%, 80%, 85%, 90%, 95% or >95% identical, with respect to its amino acid sequence, to gcdH of Pseudomonas aeruginosa, more particularly gcdH is at least 60%, 65%, 70%, 75%, 80%, 85%, 90%, 95% or >95% identical to SEQ NO 7 and has a catalytic activity of at least 75% of the activity of SEQ NO 7 and/or
   g. the protein ter is encoded by a gene derived from a strictly or facultatively anaerobic bacterium, particularly said ter is at least 60%, 65%, 70%, 75%, 80%, 85%, 90%, 95% or >95% identical, with respect to its amino acid sequence, to ter of Treponema denticola, more particularly ter is at least 60%, 65%, 70%, 75%, 80%, 85%, 90%, 95% or >95% identical to SEQ NO 8 and has a catalytic activity of at least 75% of the activity of SEQ NO 8 and/or
   h. the protein adhE1 is encoded by a gene derived from a strictly or facultatively anaerobic bacterium, particularly said adhE1 is at least 60%, 65%, 70%, 75%, 80%, 85%, 90%, 95% or >95% identical, with respect to its amino acid sequence, to adhE1 of Clostridium acetobutylicum, more particularly adhE1 is at least 60%, 65%, 70%, 75%, 80%, 85%, 90%, 95% or >95% identical to SEQ NO 9 and has a catalytic activity of at least 75% of the activity of SEQ NO 9 and/or
   i. the protein adhE2 is encoded by a gene derived from a strictly or facultatively anaerobic bacterium, particularly said adhE2 is at least 60%, 65%, 70%, 75%, 80%, 85%, 90%, 95% or >95% identical, with respect to its amino acid sequence, to adhE2 of Clostridium acetobutylicum, more particularly adhE2 is at least 60%, 65%, 70%, 75%, 80%, 85%, 90%, 95% or >95% identical to SEQ NO 13 and has a catalytic activity of at least 75% of the activity of SEQ NO 13.
14. The cell according to any one of the items 11 to 13, wherein said cell comprises the sequences for said heterologously expressed enzymes under control of a promoter sequence operable in said cell, particularly a T7 promoter, a lac promoter, a trp promoter, a tac promoter or a λP_{L} promoter.
15. A medium for n-butanol production comprising 8-12 g·L⁻¹ glucose, 8-10 g·L⁻¹ dibasic sodium phosphate dihydrate, 6-8 g·L⁻¹ monobasic potassium phosphate, 0.5-0.7 g·L⁻¹ sodium chloride, 1.2-1.5 g·L⁻¹ magnesium sulphate, 0.03-0.05 g·L⁻¹ calcium chloride dihydrate, 0.8-1.2 g·L⁻¹ ammonium chloride, and 8-12 mmol·L⁻¹ sodium bicarbonate, 0.1-0.15 μg·L⁻¹ selenium, 0.08-0.12 μg·L⁻¹ nickel, 0.7-0.9 μg·L⁻¹ molybdenum, ampicillin, spectinomycin, and kanamycin and neutral pH, particularly pH 6.8-7.3.
16. A plurality of plasmids comprising genes encoding
   a. 2-hydroxyglutarate dehydrogenase hgdH (EC 1.1.99.2.);
   b. glutaconate-CoA transferase gctAB (EC 2.8.3.12);
   c. (R)-2-hydroxyglutaryl-CoA dehydratase subunits A, B and C hgdABC (EC 4.2.1.167);
   d. glutaryl CoA dehydrogenase gcdH (EC 1.3.8.6.);
   e. trans-2-enoyl-CoA reductase (NAD+) ter (EC 1.3.1.44.); and
   f. a bifunctional aldehyde/alcohol dehydrogenase (NAD+) selected from adhE1 and adhE2 (EC 1.1.1.11/1.2.1.3.);
   particularly wherein each plasmid in said plurality of plasmids comprises more than one of said genes and each of said plasmids comprises a different selection marker, more particularly wherein the plurality of plasmids consists of three plasmids, each encoding two of said genes.
17. A plurality of plasmids according to item 16, comprising the following constructs:
   a. a plasmid comprising the genes encoding gctAB and hgdH and a gene for spectinomycin resistance and having the size of about 6.5 kbp;
   b. a plasmid comprising the genes encoding hgdABC and gcdH and a gene for kanamycin resistance and having the size of about 8.3 kbp;
   c. a plasmid comprising the genes encoding adhE1 or adhE2 and ter and a gene for ampicillin resistance and having the size of about 9.1 kbp.
18. A plurality of plasmids according to item 17, wherein said plurality comprises the following constructs:
   a. a plasmid having the sequence SEQ NO 10;
   b. a plasmid having the sequence SEQ NO 11;
   c. a plasmid having the sequence SEQ NO 12.

### BRIEF DESCRIPTION OF THE FIGURES

FIG. 1 The proposed biosynthetic pathway to produce n-butanol from 2-oxoglutarate in E. coli with indication of the reactions catalysed by the enzymes. hgdH—2-hydroxyglutarate dehydrogenase; gctAB—glutaconate-CoA transferase; hgdABC—2-hydroxyglutaryl-CoA dehydratase; gcdH—glutary-CoA dehydrogenase; ter—trans-2-enoyl-CoA reductase, adhE1/adhE2—aldehyde dehydrogenase and alcohol dehydrogenase 1 and 2.

FIG. 2 Construction of the recombinant plasmid pCDFDuet_gctAB_hgdH.

FIG. 3 Construction of the recombinant plasmid pRSFDuet_gcdH_hgdABC.

FIG. 4 Construction of the recombinant plasmid pETDuet_adhE1_ter.

FIG. 5 Construction of the recombinant plasmid pETDuet_adhE2_ter_opt

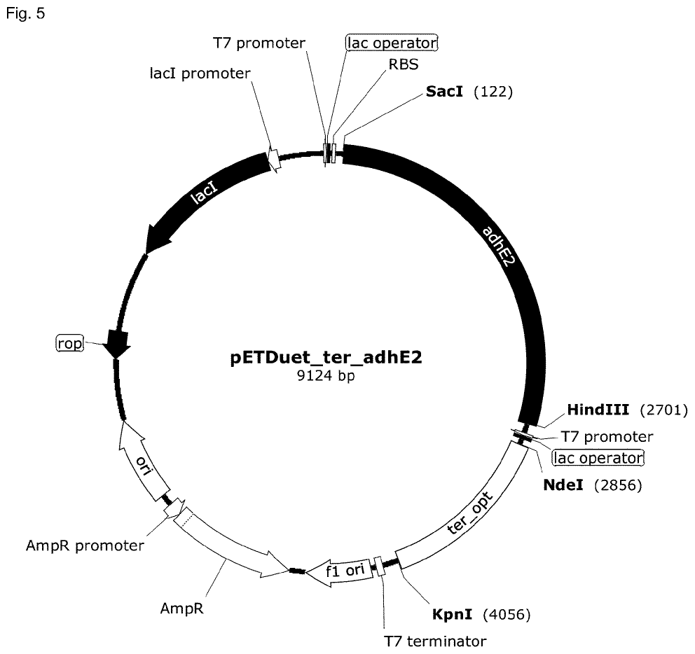

### EXAMPLES

Materials and Methods:

Cloning Procedure

E. coli NEB 5-alpha cells were used for gene cloning and vector propagation. These strains were cultured in LB medium (10 g·L⁻¹ of peptone; 5 g·L⁻¹ yeast extract and 5 g·L⁻¹ of NaCl) with the appropriate antibiotics concentration. The solid version of this medium included 15 g·L⁻¹ agar. All cultivations were performed at 37° C. and, in the case of liquid cultures, under shaking conditions (200 rpm).

For long-term storage, glycerol was added to a final concentration of 30% to overnight cultures in selective media and kept in a −80° C. freezer.

The genes used in this study were amplified by polymerase chain reaction (PCR) using Phusion High-Fidelity DNA Polymerase (Thermo Scientific, Waltham, USA) in a LifeECO Thermal Cycler (Bioer Technology, Zehjiang, China). All primers were purchased from Metabion (Munich, Germany). DNA fragments were purified using DNA Clean and Concentrator DNA Kit (Zymo Research, Irvine, USA).

Plasmids were extracted using Plasmid Miniprep kit (Zymo Research). All digestions were performed using the appropriate FastDigest® restriction endonucleases (Thermo Scientific). Ligations were performed with T4 DNA Ligase (Thermo Scientific) and transformed by heat-shock in chemically competent cells E. coli NEB 5-alpha (New England BioLabs, Massachusetts, USA). The success of ligation was checked through Colony PCR using DreamTaq (Thermo Scientific) and further confirmed by sequencing (StabVida, Lisbon, Portugal). Protocols were performed in accordance with manufacturer's instructions.

hgdH, gcdH, hgdABC and gctAB genes were codon-optimized through ATGenium for E. coli, synthesized and cloned in vector pHTPO by NZYTech (Lisbon, Portugal). A optimized codon-sequence of adhE2 and ter_opt were synthesized by ATG:biosynthetics (Freiburg, Germany) and cloned in pUC-derivative plasmids.

Plasmid Construction

Compatible vectors pETDuet, pCDFDuet and pRSFDuet (Novagen, Darmstadt, Germany) were used to provide individual expression of each protein under the control of the T7lac promoter and a ribosome-binding site (RBS).

Sources of the cloned genes are shown in table 1.

TABLE 1
Sources of n-Butanol Pathway Genes and their sequences

| Name | Abbreviation | Reference | Microorganism | Type |
|---|---|---|---|---|
| 2-Hydroxyglutarate | hgdH | EC 1.1.99.2 | Acidaminococcus | NS |
| dehydrogenase | | NCBI GI: | fermentans | AS |
| | | >gb\|CP001859.1\|:1104274- | ATCC 25085 | NS CO |
| | | 1105269 | | |
| | | NCBI | | |
| | | GeneID: Acfer_0977 | | |
| Glutaconate-CoA | gctAB | EC 2.8.3.12 | Acidaminococcus | NS (A) |
| transferase | | gctA | fermentans | AS (A) |
| | | NCBI GI | ATCC 25085 | NS CO (A) |
| | | >gi\|284047386:2019003- | | NS (B) |
| | | 2019965 | | AS (B) |
| | | NCBI GeneID: Acfer_1820 | | NS CO (B) |
| | | gctB | | |
| | | NCBI GI: | | |
| | | >gi\|284047386:2018200- | | |
| | | 2019000 | | |
| | | NCBI geneID: Acfer_1819 | | |
| (R)-2-hydroxyglutaryl- | hgdAB | EC 4.2.1.- | Clostridium | NS (A) |
| CoA dehydrogenase | | NCBI GI: | symbiosum | AS (A) |
| subunits A and B | | >AF123384.1:1241-2683 | ATCC 14940 | NS CO (A) |
| | | NCBI geneID: AF123384 | | NS (B) |
| | | | | AS (B) |
| | | | | NS CO (B) |
| (R)-2-hydroxyglutaryl- | hgdC | EC 4.2.1.167 | Acidaminococcus | NS (C) |
| CoA dehydrogenase | | NCBI GI: | fermentans | AS (C) |
| Subunit C | | >gi\|284047386:2015608- | ATCC 25085 | NS CO (C) |
| | | 2016390 | | |
| | | NCBI GeneID: | | |
| | | Acfer_0168 | | |
| Glutaryl-CoA | gcdH | EC 1.3.8.6 | Pseudomonas | NS |
| dehydrogenase | | NCBI GI: >NP_249138.1 | aeruginosa | AS |
| | | NCBI | PAO1 | NS CO |
| | | GeneID: PANN_05040 | | |
| trans-2-enoyl-CoA | ter | EC 1.3.1.44 | Treponema | NS |
| reductase (NAD⁺) | | NCBI GI: | denticola. | AS |
| | | >AE017226.1:636109- | ATCC 35405 | |
| | | 637302 | | |
| | | NCBI GeneID: TDE_0597 | | |
| Bifunctional Aldehyde/ | adhE | EC 1.1.1.11/1.2.1.3 | Clostridium | NS |
| Alcohol | | NCBI GI: | acetobutylicum | AS |
| dehydrogenase | | >CP002661.1:33722- | pSMBa- | |
| (NAD⁺) | | 36298 | DSM 1731 | |
| | | NCBI GeneID: adhE | | |
| | | ATCC ID: DSM 1731 | | |

A—alpha subunit;
B—beta subunit B;
C—gamma subunit C;
NS—Nucleic Acid Sequence;
AS—Amino acid Sequence;
CO—Codon Optimized

The plasmid pCDFDuet (Novagen) was used to clone the codon-optimized genes encoding the first two reactions of the proposed pathway (gctAB and hgdH.). hgdH was amplified using the primers hgdH fw and hgdH rev with flanking restriction sites for KpnI and XhoI and cloned into pCDFDuet. The PCR product for gctAB, amplified using primers gctAB_fw and gctAB_rev, was restricted and ligated into BamHI and HindIII restriction sites of the previous construction. Colony PCR with appropriate primers was used to find successful clones and the final plasmid was sent for sequencing to confirm the sequence was correct.

The plasmid pRSFDuet (Novagen) was used to clone the codon optimized genes hgdABC and gcdH, corresponding to the two intermediate steps of the proposed pathway. gcdH was amplified using the primers gcdH fw and gcdH rev with restriction sites to NdeI and XhoI and cloned in pRSFDuet. Then, hgdABC was inserted in the previous construction. This gene was amplified using primers hgdABC fw and hgdABC rev with restriction sites for SacI and NotI, respectively. Colony PCR with appropriate primers was used to find successful clones and the final plasmid was sent for sequencing to confirm the sequence was correct.

The plasmid pETDuet (Novagen) was used to clone the genes adhE1 and ter, corresponding to the last two genes of the proposed pathway. The adhE1 gene was amplified from template plasmid pmTA1 (Nielsen, et al. (2009), Metabolic engineering. Elsevier, 11(4-5), pp. 262-73.) using primers adhE1_fw and adhE1_rev with restriction sites for EcoRI and NotI, respectively. The synthetic gene ter (ATG:biosynthetics, Freiburg, Germany) was amplified using primers ter fw and ter rev; restricted and ligated into NdeI and XhoI restriction sites of the previous construction pETDuet_adhe1, resulting in the plasmid pETDuet_adhE1_ter. Colony PCR with appropriate primers was used to find successful clones and the final plasmid was sent for sequencing to confirm the sequence was correct.

Finally, the plasmid pETDuet (Novagen) was used to clone the genes adhE2 and ter_opt, corresponding to the last two genes of the proposed pathway. The codon-optimized synthetic gene ter_opt (ATG:biosynthetics, Freiburg, Germany) gene was directly digested with NdeI and KpnI and cloned in the respective restriction sites of pETDuet. The codon-optimized synthetic gene adhE2 (ATG:biosynthetics, Freiburg, Germany) was restricted and ligated into SacI and HindIII restriction sites of the previous construction pETDuet_ter, resulting in the plasmid pETDuet_adhE2_teropt.

In Table 2, the primers used in this study for PCR amplification are shown.

TABLE 2
Sequences of primers used in the cloning procedures
of this study (*restriction sites are underlined).
fw—forward; rev—reverse

| | | SEQ | Restriction |
|---|---|---|---|
| Primer | Sequence | NO | Sites* |
| adhE1_fw | CCGAATTCATGAAAGTCACAACAGTAAAGG | 17 | EcoRI |
| adhE1_rev | CCGCGGCCGCTTAAGGTTGTTTTTTAAAACAATT | 18 | NotI |
| ter_fw | CCCATATGATTGTAAAACC | 19 | NdeI |
| ter_rev | CCCTCGAGTTAAATC | 20 | XhoI |
| hgdABC_fw | CCGAGCTCATGAGTATCTATACCCTGGGC | 21 | SacI |
| hgdABC_rev | CCGCGGCCGCTTATTTTTGCATCTCCAAAAC | 22 | NotI |
| gcdH_fw | CCCATATGGCAACCAAAGCAAG | 23 | NdeI |
| gcdH_rev | CCCTCGAGTCAAAAGAACGCTTGAATACC | 24 | XhoI |
| hgdH_fw | CCGGTACCATGAAAGTGCTGTGCTACGG | 25 | KpnI |
| hgdH_rev | CCCTCGAGTTATTTGATTTTGTTCGGGC | 26 | XhoI |
| gctAB_fw | CCGGATCCATGAGCAAAGTCATGACCC | 27 | BamHI |
| gctAB_rev | CCAAGCTTTTATTTGGCTTCAGTTGGAAC | 28 | HindIII |

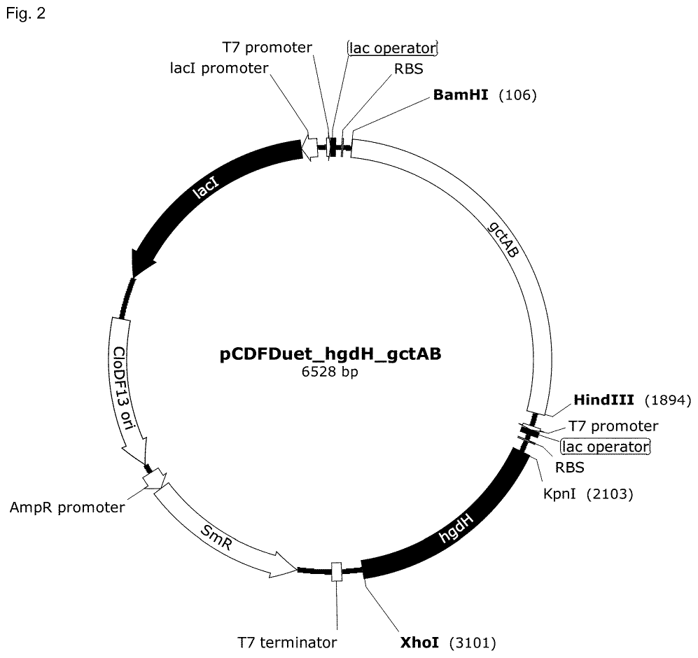

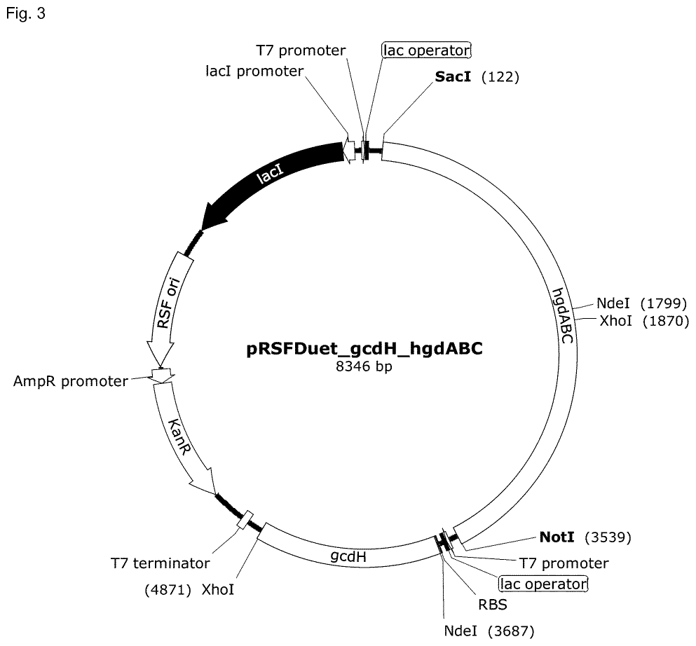

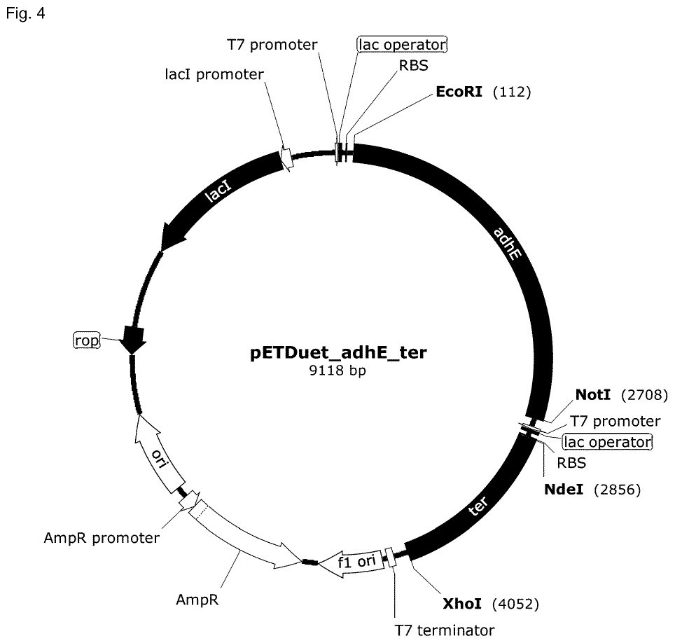

The success of the plasmid constructions was confirmed by sequencing the regions of interest with the appropriate primers. In FIG. 2-4 and table 3 the plasmids used or constructed in this study, as well as the respective major features are shown.

TABLE 3
Plasmids used in this study

| Plasmid | Construct | Source |
|---|---|---|
| pETDuet | ColE1(pBR322) ori, lacl, double T7lac, AmpR | Novagen |
| pCDFDuet | CloDF13 ori, lacl, double T7lac, StrepR | Novagen |
| pRSFDuet | RSF ori, lacl, double T7lac, KanR | Novagen |
| pETDuet_adhE1_ter | pETDuet carrying adhE1 from C. acetobutylicum | This study |
| | and ter from T. denticola | |
| pCDFDuet_gctAB_hgdH | pCDFDuet carrying codon-optimized gctAB and | This study |
| | hgdH from A. fermentans | |
| pRSFDuet_gcdH_hgdABC | pRSFDuet carrying codon-optimized hgdC from | This study |
| | A. fermentans; hgdAB from Clostridium | |
| | symbiosum and gcdH from Pseudomonas | |
| | aeruginosa | |
| pETDuet_adhe2_ter_opt | pETDuet carrying codon-optimized adhE2 from | This study |
| | C. acetobutylicum and ter from T. denticola | |
| pmTA1 | Gmr, lacl, taclac: thil, adhE | Nielsen, D. R. |
| | | et al. (2009) |
| | | Elsevier, 11(4- |
| | | 5), pp. 262-73. |

Bacterial Strains

E. coli K12 MG1655 (DE3) and E. coli BL21 (DE3) were used as hosts for gene expression under control of T7 promoter. BUT_OXG1 and BUT_OXG2 strains were obtained by transforming E. coli BL21 (DE3) and E. coli K12 MG1655 (DE3), respectively, with pCDFDuet_gctAB_hgdH; pRSFDuet_gcdH_hgdABC and pETDuet_adhE1_ter by electroporation. The control strains Control_OXG1 and Control_OXG2 were obtained, by transforming, respectively, E. coli BL21 (DE3) and E. coli K12 MG1655 (DE3) with the plasmids expressing only the last five enzymes of the pathway (pRSFDuet_gcdH_hgdABC and pETDuet_adhE1_ter). Electrocompetent cells were prepared using the protocol developed by (Dower, et al. (1988), Nucleic Acids Research, 16(13), pp. 6127-6145) and transformed using 0.1 cm-gap electroporation cuvettes at a voltage of 1.8 KV. Positive transformants were isolated in LB (containing 10 g·L⁻¹ of peptone; 5 g·L⁻¹ yeast extract and 5 g·L⁻¹ of NaCl) agar (15 g·L⁻¹) plates, containing the appropriate antibiotic concentrations (50 μg·mL⁻¹ ampicillin, 50 μg·mL⁻¹ spectinomycin and 30 μg·mL⁻¹ kanamycin) and incubated at 37° C., overnight. To confirm the success of the transformation, a few transformant colonies were cultivated in LB medium with antibiotics, overnight. After, plasmids were extracted and digested with appropriate restriction enzymes. The correct fragment lengths were confirmed by running the digestion in a 1% (w/v) agarose gel.

BUT_OXG3 was constructed in the same fashion described above but expressing codon-optimized sequences of ter from Treponoema denticola and adhE2 from Clostridium acetobutylicum.

TABLE 4
List of strains and genomic DNA used or engineered for this study

| Strains | Relevant genotype | Source |
|---|---|---|
| E. coli K12 MG1655 | F-λ-ilvGrfb-50 rph-1 λ(DE3) | Nielsen, et al. |
| (DE3) | | (2009), Metabolic |
| | | engineering. |
| | | Elsevier, 11(4-5), |
| | | pp. 262-73.) |
| E. coli BL21 (DE3) | fhuA2 [lon] ompT gal (λ DE3) [dcm] ΔhsdS | New England |
| | λ DE3 = λ sBamHlo ΔEcoRI-B | Labs |
| | int::(lacl::PlacUV5::T7 gene1) i21 Δnin5 | |
| BUT_OXG1 | E. coli BL21 DE3 pETDuet_adhE1_ter; | This study |
| | pCDFDuet_gctAB_hgdH; pRSFDuet_gcdH_hgdABC | |
| BUT_OXG2 | E. coli K12 MG1655 DE3 pETDuet_adhE1_ter; | This study |
| | pCDFDuet_gctAB_hgdH; pRSFDuet_gcdH_hgdABC | |
| Control_OXG1 | E. coli BL21 DE3 pETDuet_adhE1_ter; | This study |
| | pRSFDuet_gcdH_hgdABC | |
| Control_OXG2 | E. coli K12 MG1655 DE3 pETDuet_adhE1_ter; | This study |
| | pRSFDuet_gcdH_hgdABC | |
| BUT_OXG3 | E. coli K12 MG1655 DE3 pETDuet_adhE2_ter_opt; | This study |
| | pCDFDuet_gctAB_hgdH; pRSFDuet_gcdH_hgdABC | |

Table 4 summarizes the strains of E. coli used or engineered for this study.

Enzymatic Assays

Table 5 lists the enzymatic reactions and table 6 lists the enzymatic assays for the heterologous enzymes in n-butanol production. The stated reactions are the basis for any reactivity quantities stated herein, unless explicitly stated otherwise.

TABLE 5
Enzymatic reactions.

| | | | | Equation |
|---|---|---|---|---|
| Reaction | Enzyme | EC No. | Code | number |
| 2-oxoglutarate + NADH + H⁺ = (S)-2- | 2-hydroxyglutarate | 1.1.99.2 | hgdH | Eq. i |
| hydroxyglutarate + NAD⁺ | dehydrogenase | | | |
| | (NADH) | | | |
| acetyl-CoA + (S)-2-hydroxyglutarate = acetate + | glutaconate CoA- | 2.8.3.12 | gctAB | Eq. ii |
| (R)-2-hydroxyglutaryl-CoA | transferase | | | |
| (R)-2-hydroxyglutaryl-CoA = (E)-glutaconyl- | (R)-2-hydroxyglutaryl- | 4.2.1.167 | hgdABC | Eq. iii |
| CoA + H₂O | CoA dehydratase | | | |
| (E)-glutaconyl-CoA = crotonyl-CoA + CO₂ | glutaryl-CoA | 1.3.8.6 | gcdH | Eq. iv |
| | dehydrogenase | | | |
| | (ETF) | | | |
| crotonyl-CoA + NADH + H⁺ = butanoyl-CoA + | trans-2-enoyl-CoA | 1.3.1.44 | ter | Eq. v |
| NAD⁺ | reductase (NAD⁺) | | | |
| butanal + NAD⁺ + H₂O = butanoyl-CoA + | aldehyde | 1.2.1.3 | adhE | Eq. vi |
| NADH + H⁺ | dehydrogenase | | | |
| | (NAD⁺) | | | |
| n-butanol + NAD⁺ = butanal + NADH + H⁺ | alcohol | 1.1.1.1 | adhE | Eq. vii |
| | dehydrogenase | | | |
| | (NAD⁺) | | | |

The above named compound are also referred to as the following synonyms:

2-oxoglutarate: 2-Oxopentanedioic acid; 2-Ketoglutaric acid; alpha-Ketoglutaric acid; 2-Oxoglutaric acid; Oxoglutaric acid; 2-oxopentanedioate; 4-carboxy-2-oxobutanoate; 2-ketoglutarate; 2-oxopentanedioic acid; α-ketoglutarate.

2-hydroxyglutarate: 2-hydroxypentanedioate.

2-hydroxyglutaryl-CoA: 3′-phosphoadenosine 5′-{3-[(3R)-4-{[3-({2-[(4-carboxy-2-hydroxybutanoyl)sulfanyl]ethyl}amino)-3-oxopropyl]amino}-3-hydroxy-2,2-dimethyl-4-oxobutyl] dihydrogen diphosphate} 2-hydroxyglutaryl-coenzyme A.

Glutaconyl-CoA: 5-[2-[3-[[4-[[[5-(6-aminopurin-9-yl)-4-hydroxy-3-phosphonooxyoxolan-2-yl]methoxy-hydroxyphosphoryl]oxy-hydroxyphosphoryl]oxy-2-hydroxy-3,3-dimethylbutanoyl]amino]propanoylamino]ethylsulfanyl]-5-oxopent-3-enoic acid

Crotonyl-CoA: E)-but-2-enoyl-CoA; Crotonoyl-CoA; trans-But-2-enoyl-CoA; trans-butyr-2-enoyl-CoA.

Butanoyl-CoA: butyryl-CoA; butanoyl-coenzyme A; Butyryl-coenzyme A.

Butanal: Butyraldehyde; 1-Butanal; Butaldehyde; Butyl aldehyde; n-Butanal.

n-Butanol: Butan-1-ol; Butalcohol; Butanol; 1-Butanol; Butyl alcohol; Butyl hydrate; Butylic alcohol; Butyralcohol; Butyric alcohol; Butyryl alcohol; n-Butyl alcohol; 1-Hydroxybutane; n-Propylcarbinol; 1-butyl alcohol.

TABLE 6
Enzymatic Assays used for individual enzyme
activities used in pathway for n-butanol production

| Enzyme | EC No. | Assay Reference |
|---|---|---|
| 2-hydroxyglutarate | 1.1.99.2 | Kranendijk M. et al., J. Inherit |
| dehydrogenase | | Metab. Dis. (2009), 32(6):713-19. |
| glutaconate CoA- | 2.8.3.12 | Charrier C. et al. Microbiology |
| transferase | | (2006), 152, 179-185. |
| (R)-2-hydroxyglutaryl- | 4.2.1.167 | Schweiger G. et al. Arch. |
| CoA dehydratase | | Microbiol. (1984), 137:302-307. |
| glutaryl-CoA | 1.3.8.6 | Estelmann S. et al. FEBS J. |
| dehydrogenase (ETF) | | (2014( ), 4:5120-5131. |
| trans-2-enoyl-CoA | 1.3.1.44 | Bond-Watts B. et al. Nature Chem. |
| reductase (NAD⁺) | | Biol. (2011), 7:22-27. |
| aldehyde dehydrogenase | 1.2.1.3 | Guro S. et al. Alcohol. (1990), |
| (NAD⁺) | | 7(5):397-401. |
| alcohol dehydrogenase | 1.1.1.1 | Guro S. et al. Alcohol. (1990), |
| (NAD⁺) | | 7(5):397-401. |

The above named enzymes are also referred to as the following synonyms:

2-hydroxyqlutarate dehydrogenase: L-2-hydroxyglutarate dehydrogenase; L-alpha-hydroxyglutarate dehydrogenase; alpha-hydroxyglutarate dehydrogenase; alpha-hydroxyglutarate oxidoreductase; (S)-2-hydroxyglutarate:acceptor 2-oxidoreductase; alpha-ketoglutarate reductase; hydroxyglutaric dehydrogenase; L-2-hydroxyglutaric acid dehydrogenase.

glutaconate CoA-transferase: (E)-glutaconate CoA-transferase; glutaconate CoA-transferase; Acetyl-CoA:(E)-glutaconate CoA-transferase.

(R)-2-hydroxyglutaryl-CoA dehydratase: (R)-2-hydroxyglutaryl-CoA hydro-lyase ((E)-glutaconyl-CoA-forming).

glutaryl-CoA dehydrogenase: glutaryl-coenzyme A dehydrogenase; Glutaryl-CoA dehydrogenase.

trans-2-enoyl-CoA reductase: mitochondrial 2-trans-enoyl-CoA/ACP reductase; NADPH-dependent trans-2-enoyl-CoA reductase; 2-trans enoyl-ACP(CoA) reductase; trans-2-enoyl-CoA reductase (NADPH); mitochondrial 2-trans-enoyl-thioester reductase.

bifunctional aldehyde/alcohol dehydrogenase: aldehyde dehydrogenase; aldehyde reductase; aldehyde/alcohol dehydrogenase; aliphatic alcohol dehydrogenase; ethanol dehydrogenase; NAD+-dependent alcohol dehydrogenase; NAD-dependent alcohol dehydrogenase; NAD-specific aromatic alcohol dehydrogenase; NADH-alcohol dehydrogenase; NADH-aldehyde dehydrogenase; NADH-dependent alcohol dehydrogenase.

Butanol Production Experiments in Complex Medium

The strains BUT_OXG1 and BUT_OXG2 were cultivated in Terrific Broth (TB) medium supplemented with glucose, glutamate, riboflavin and iron (III) citrate according to composition shown in table 7. The pH of this medium was 7.2±0.2 at 25° C.

TABLE 7
Medium composition of Terrific Broth.

| Component | Amount per Liter | Unit |
|---|---|---|
| Tryptone | 12 | g |
| Yeast extract | 24 | g |
| Glycerol | 4 | mL |
| Monobasic potassium phosphate | 2.31 | g |
| Dibasic potassium phosphate | 12.54 | g |
| Glutamate | 0.468 | g |
| Riboflavin | 0.07529 | g |
| Iron (III) citrate | 0.525 | g |
| Glucose | 10 | g |

A single colony was picked from Luria-Bertani (LB) plates and inoculated in 10 mL of LB medium (Table 8).

TABLE 8
LB medium composition.

| Component | Amount per Liter | Unit |
|---|---|---|
| Tryptone | 10 | g |
| Yeast extract | 5 | g |
| Sodium Chloride | 10 | g |

Cultivation was performed with the addition of suitable antibiotics according to the employed plasmids (50 μg·mL⁻¹ ampicillin, 50 μg·mL⁻¹ spectinomycin, and 30 μg·mL⁻¹ kanamycin). The pre-cultures were grown aerobically on a rotary shaker at 37° C. and 200 rpm, overnight.

500 mL shake flasks with 100 mL of TB medium, containing appropriate antibiotics, were inoculated with pre-cultures to obtain an initial optical density OD₆₀₀ of 0.1. Cultivation was carried on a rotary shaker at 200 rpm at 37° C. The butanol production genes were induced by the addition of 0.1, 0.5 or 1 mmol·L⁻¹ isopropyl 1-thio-μ-D-galactopyranoside (IPTG) to the culture medium when an optical density OD₆₀₀ of 0.4-0.5 was reached.

To promote butanol production, after induction, the cells were switched to anaerobic conditions by transferring 60 mL of culture to 120 mL sealed serum flasks. The culture was supplemented with 600 μL of a 0.01 M stock solution of sodium bicarbonate to achieve a final concentration of 10 mmol·L⁻¹, since it reduces long lag phases in E. coli anaerobic growth (Hornsten (1995), Bioprocess Engineering, 12, pp. 157-162.).

The cultures were incubated at 30° C. and 180 rpm, for 96 hours. Samples of culture broth were collected at time 0, during induction time and at 96 h. All the experiments were performed in triplicate and the samples were analysed by High-Performance Liquid Chromatography (HPLC) and Gas Chromatography (GC).

Butanol Production Experiments in Defined Medium

The strains BUT_OXG1 and BUT_OXG2 were cultivated in High Density Medium (HDM) adapted from (Sivashanmugam, A. et al. (2009), 18(1), pp. 936-948.), supplemented with a solution of amino acids, extra glutamate, riboflavin and iron citrate (III), according to table 9. The pH of the medium was adjusted to 7.1 using 2 mol·L⁻¹ NaOH.

TABLE 9
Medium composition of HDM,
adapted from (Sivashanmugam (2009) ibid)

| Component | Amount per Liter | Unit |
|---|---|---|
| Glucose | 10 | g |
| Dibasic sodium phosphate dihydrate | 8.89 | g |
| Monobasic potassium phosphate | 6.8 | g |
| Sodium chloride | 0.58 | g |
| Magnesium sulphate | 1.35 | g |
| Calcium chloride dihydrate | 0.038 | g |
| Ammonium chloride | 1 | g |
| Trace metals | 250 | μL |
| Vitamins BME100x | 250 | μL |
| Amino acid mix | 2 | g |
| Glutamate | 0.468 | g |
| Riboflavin | 0.07529 | g |
| Iron (III) citrate | 0.525 | g |

The trace metals solution contained (per liter): FeSO₄.7H₂O (30 mg); ZnSO₄.7H₂O (45 mg); CaCl₂.2H₂O (45 mg); MnCl₂.2H₂O (100 mg); CoCl₂.6H₂O (30 mg); CuSO₄.5H₂O (30 mg); Na₂MoO₄.2H₂O (40 mg); H₃BO₃ (10 mg); KI (10 mg) and Na₂EDTA (1.5 g). The amino acid mix contained 1 g of adenine and 4 g of arginine, aspartate, glutamate, histidine, isoleucine, lysine, methionine, phenylalanine, serine, threonine, tryptophan, tyrosine and valine. The vitamin BME 100× solution (Sigma Aldrich, St. Louis, Mo., USA) contained (per liter): D-biotin (0.1 g); choline chloride (0.1 g); folic acid (0.1 g); myo-inositol (0.2 g); niacinamide (0.1 g); D-pantothenic acid. % Ca (0.1 g); riboflavin (0.01 g); thiamine.HCl (0.1 g) and NaCl (8.5 g).

For the pre-cultures, a single colony was picked from Luria-Bertani (LB) plates and inoculated in 10 mL of LB medium. Cultivation was performed with the addition of suitable antibiotics according to the employed plasmids (50 μg·mL⁻¹ ampicillin, 50 μg·mL⁻¹ spectinomycin, and 30 μg·mL⁻¹ kanamycin). The pre-cultures were grown aerobically on a rotary shaker at 37° C. and 200 rpm, overnight. Cells were washed and harvested by centrifugation (10 min at 3000×g). Afterwards, an appropriate volume of pre-culture was transferred to 500 mL shake flasks with 100 mL of HDM medium, containing the appropriate antibiotics, yielding an initial OD₆₀₀ of 0.1. This culture was cultivated on a rotary shaker at 200 rpm at 37° C. The butanol production genes were induced with 0.1, 0.5 or 1 mmol·L⁻¹ isopropyl 1-thio-β-D-galactopyranoside (IPTG) at an OD₆₀₀ of 0.4-0.5.

After induction, 60 mL of the culture were transferred to 120 mL sealed serum flasks to promote butanol production under anaerobic conditions. The culture was supplemented with 600 μL of a 0.01 mol·L⁻¹ stock solution of sodium bicarbonate to achieve a final concentration of 10 mmol·L⁻¹ (to reduce lag phases in E. coli anaerobic growth (Hornsten, 1995, Bioprocess Engineering, 12, pp. 157-162)). Selenium, nickel and molybdenum are part of the formate hydrogen lyase (FHL) complex, which is induced under anaerobic conditions. For this reason, 60 μL of a solution of extra trace metals (NiCl₂(1.7 mg·L⁻¹); (NH₄)₆Mo₇O₂₄ (14.5 mg·L⁻¹); 4H₂O Na₂SeO₃ (2.4 mg·L⁻¹)) was supplied to the medium.

The cultures were incubated at 30° C. and 180 rpm, for 96 hours. Samples of supernatant were collected at time 0, induction time and 96 h. All the experiments were performed in triplicate and the samples were analysed by GC.

Analytical Methods

Samples were centrifuged at 6000×g for 10 min to separate cells from the medium. Afterwards, the supernatant was filtered with a 0.22 μm pore filter membrane to glass vials and stored at −20° C. until analysed.

Butanol concentration was quantified by a Gas Chromatograph GP-9000 system (Chrompack) with a Meta-WAX capillary column (30 m×0.25 mm×0.25 μm) equipped with a flame ionization detector (FID); helium was used as carrier gas with a flow rate of 1 mL·min⁻¹. The filtered supernatant (900 μL) was mixed with 100 μL of a 5 g·L⁻¹ solution of isobutanol, the internal standard, yielding a final concentration of 0.5 g·L⁻¹, and 1 μL of this mixture was injected. The temperature of injector and detector were maintained at 250° C. The column was initially at 50° C., heated to 177.5° C. at a 5° C.·min⁻¹ rate and then heated to 230° C. at 10° C.·min⁻¹, which was held for 15 minutes. A calibration curve was obtained by injecting standards with several concentrations of butanol and a fixed concentration of internal standard (0.5 g·L⁻¹ of isobutanol). Butanol concentration was calculated by comparing the ratio between its peak area and internal standard peak area with calibration curves.

All cell optical density measurements at 600 nm (OD₆₀₀) were performed using the spectrophotometer Ultrospec 10 from Biochrom (Cambridge, UK).

### Example 1

Preparation of a n-Butanol Producing Microbial Organism Having a Pathway Coupling the Enzymes Glutaryl-CoA Dehydrogenase and Trans-2-Enoyl-CoA Reductase in Complex Medium.

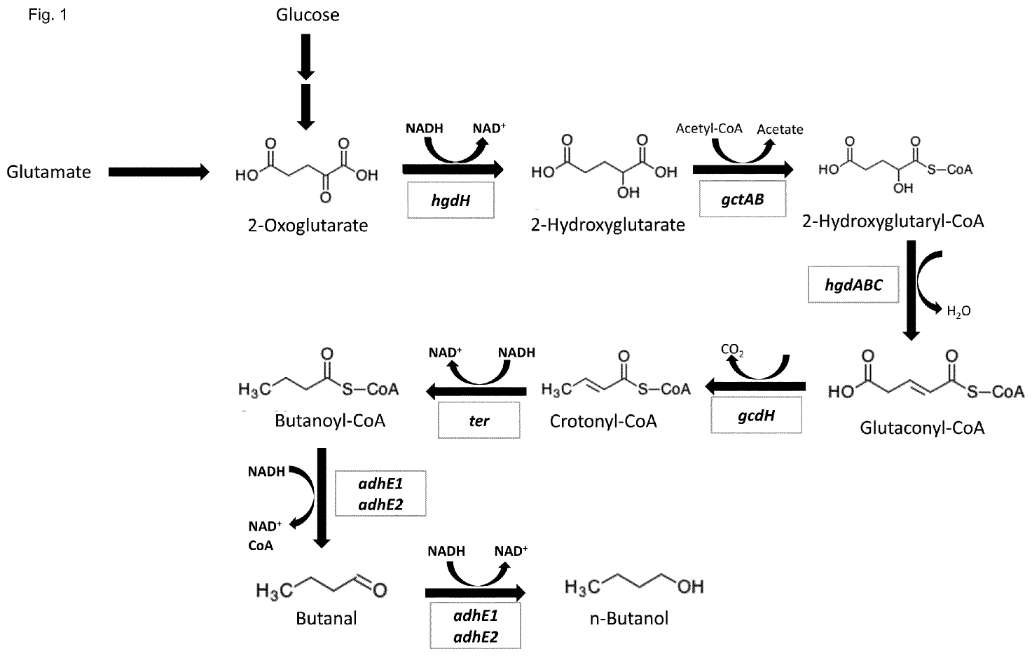

This example describes the generation of a microbial organism capable of producing n-butanol from 2-oxoglutarate in complex medium. Escherichia coli is used as target organism to engineer the butanol pathway shown in FIG. 1, where glutaryl-CoA dehydrogenase activity was coupled to enzymes activities of 2-hydroxyglutarate dehydrogenase, glutaconate-CoA transferase, 2-hydroxyglutaryl-CoA dehydratase, trans-2-enoyl-CoA reductase, aldehyde dehydrogenase and alcohol dehydrogenase. The resulting genetically engineered strains of E. coli, BUT_OXG1 and BUT_OXG2, were used for butanol production by cultivation in Terrific Broth (TB) medium. Butanol production 96 h after inoculation is shown in Table 10.

TABLE 10
Butanol production in TB medium 96 h after inoculation.
Butanol (mg · L⁻¹)

| IPTG (mmol · L⁻¹) | BUT_OXG1 | BUT_OXG2 |
|---|---|---|
| 1 | 4.5 ± 0.1 | 16.07 ± 2.3 |
| 0.5 | 6.8 ± 0.46 | 24.05 ± 4.6 |
| 0.1 | 3.2 ± 0.05 | 7.25 ± 0.8 |

### Example 2

Preparation of a Producing Microbial Organism Having a Pathway Coupling the Enzymes Glutaryl-CoA Dehydrogenase and Trans-2-Enoyl-CoA Reductase Capable to Produce n-Butanol from 2-Oxoglutarate in Defined Medium.

This example describes the generation of a microbial organism capable of producing butanol from 2-oxoglutarate in a defined medium. Escherichia coli is used as target organism to engineer the butanol pathway shown in FIG. 1, where glutaryl-CoA dehydrogenase activity was coupled to enzymes activities of 2-hydroxyglutarate dehydrogenase, glutaconate-CoA transferase, 2-hydroxyglutaryl-CoA dehydratase, trans-2-enoyl-CoA reductase, aldehyde dehydrogenase and alcohol dehydrogenase. Butanol production 96 h after inoculation is shown in Table 11.

TABLE 11
Butanol production in defined medium 96 h after inoculation.
Butanol (mg · L⁻¹)

| IPTG (mmol · L⁻¹) | BUT_OXG1 | BUT_OXG2 |
|---|---|---|
| 1 | 7.0 ± 0.59 | 59.95 ± 6.14 |
| 0.5 | 29.04 ± 1.72 | 75.32 ± 4.21 |
| 0.1 | 5.6 ± 0.05 | 20.96 ± 2.86 |

### Example 3

Negative Control for the n-Butanol Producing Microbial Organism Having a Pathway where Glutaryl-CoA Dehydrogenase Activity was Coupled Only to Enzymes Activities of 2-Hydroxyglutaryl-CoA Dehydratase, Trans-2-Enoyl-CoA Reductase, Alcohol Dehydrogenase and Aldehyde Dehydrogenase.

This example describes the generation of a microbial organism incapable of producing butanol from 2-oxoglutarate. This example is considered as negative control since the absence of coupled enzymes will lead to an n-butanol unproductive microbial organism. Butanol production 96 h after inoculation is shown in Table 12. The method detection limit is 3 mg·L⁻¹.

TABLE 12
Butanol production in TB and defined medium 96 h
after inoculation from a strain lacking hgdH and gctAB.

| | Butanol final titer (mg · L⁻¹) | |
|---|---|---|
| Strain | TB | HDM |
| Ct_OXG1 | n.d. | n.d. |
| Ct_OXG2 | n.d. | n.d. |

n.d.: not detectable.

### Example 4

Preparation of a n-Butanol Producing Microbial Organism Having a Pathway where Glutaryl-CoA Dehydrogenase Activity was Coupled to Enzymes Activities of 2-Hydroxyglutaryl-CoA Dehydratase, Trans-2-Enoyl-CoA Reductase, Alcohol Dehydrogenase and Aldehyde Dehydrogenase in Aerobic Conditions.

This example describes the generation of a microbial organism incapable of producing butanol from 2-oxoglutarate. This example is considered as negative control since the absence of anaerobic conditions will lead to an n-butanol unproductive microbial organism. Butanol production 96 h after inoculation is shown in Table 13. The method detection limit is 3 mg. L⁻¹.

TABLE 13
Butanol production under aerobic conditions
in TB and HDM medium 96 h after inoculation.

| | Butanol final titer (mg · L⁻¹) | |
|---|---|---|
| Strain | TB | HDM |
| BUT_OXG1 | n.d. | n.d. |
| BUT_OXG2 | n.d. | n.d. |

n.d.: not detectable.

### Example 5

Optimizing the n-Butanol Production

Three factors were changed to increase n-butanol production.

In the first task, the switch to serum bottles was delayed by 4 and 12 h after IPTG induction. By doing so, the butanol titer was increased by 1.6-fold (to 129±8 mg·L⁻¹ for 12 h delay).

Secondly, alcohol dehydrogenase 1 (adhE1) was replaced by alcohol dehydrogenase 2 (adhE2). Reportedly, the protein-product of adhE2 has more activity in E. coli. A codon-optimized sequence of the adhE2 gene (WT: SEQ NO 14, codon-optimized: SEQ NO 15) and of the gene encoding the trans-2-enoyl-reductase (ter) (SEQ NO 16) were cloned and expressed in E. coli obtaining BUT_OXG3 strain. These two genes were the only ones that were not codon-optimized in the previous engineered strains. The maximum butanol titer obtained in the experiments with the codon-optimized strains was 172±2 mg·L⁻¹ (with a switch to serum bottles 12 h after IPTG induction).

Thirdly, the medium was supplemented with extra glutamate (2 g·L⁻¹) at the anaerobic switch moment. The conditions of this last experiment were the following: the working volume was reduced from 60 mL to 40 mL and the switch to anaerobic conditions was 4 h after the IPTG induction. The maximum butanol titer obtained in this experiment was 187±2 mg·L⁻¹.

1. A method for production of n-butanol, wherein a transgenic cell heterologously expressing each of the following enzymes:
a. 2-hydroxyglutarate dehydrogenase hgdH (EC 1.1.99.2.);
b. glutaconate-CoA transferase gctAB (EC 2.8.3.12);
c. (R)-2-hydroxyglutaryl-CoA dehydratase subunits A, B and C hgdABC (EC 4.2.1.167);
d. glutaryl CoA dehydrogenase gcdH (EC 1.3.8.6.);
e. trans-2-enoyl-CoA reductase (NAD+) ter (EC 1.3.1.44.); and
f. a bifunctional aldehyde/alcohol dehydrogenase (NAD+) selected from adhE1 and adhE2 (EC 1.1.1.11/1.2.1.3.);
is grown in a medium comprising a metabolic precursor of 2-oxoglutarate.

2. The method according to claim 1, wherein n-butanol is extracted from said medium.

3. The method according to claim 1, wherein said metabolic precursor of 2-oxoglutarate is selected from glucose, glycerol, glutamate or acetate.

4. The method according to claim 1, wherein the transgenic cell is a bacterium or a yeast cell.

5. The method according to claim 4, wherein the bacterium or the yeast cell is selected from genera Escherichia, Corynebacterium, Ralstonia, Clostridium, Pseudomonas, Lactobacillus, Lactococcus, Acidaminococcus, Fusobacterium, Peptoniphilus, Saccharomyces, Streptomyces Lactobacillus, Pichia, Kluyveromyces, Yarrowia, or Staphylococci, particularly Escherichia coli.

6. The method according to claim 1, wherein
a. the protein hgdH is encoded by a gene derived from a strictly or facultatively anaerobic bacterium, particularly said hgdH is at least 60%, 65%, 70%, 75%, 80%, 85%, 90%, 95% or >95% identical, with respect to its amino acid sequence, to hgdH of Acidaminococcus fermentans, more particularly hgdH is at least 60%, 65%, 70%, 75%, 80%, 85%, 90%, 95% or >95% identical to SEQ NO 1 and has a catalytic activity of at least 75% of the activity of SEQ NO 1 and/or
b. the protein gctAB is encoded by a gene derived from a strictly or facultatively anaerobic bacterium, particularly said gctAB is at least 60%, 65%, 70%, 75%, 80%, 85%, 90%, 95% or >95% identical, with respect to its amino acid sequence, to gctAB of Acidaminococcus fermentans, more particularly subunit A of gctAB is at least 60%, 65%, 70%, 75%, 80%, 85%, 90%, 95% or >95% identical to SEQ NO 2 and has a catalytic activity of at least 75% of the activity of SEQ NO 2 and/or subunit B of gctAB is at least 60%, 65%, 70%, 75%, 80%, 85%, 90%, 95% or >95% identical to SEQ NO 3 and has a catalytic activity of at least 75% of the activity of SEQ NO 3 and/or
c. the A subunit of the protein hgd is encoded by a gene derived from a strictly or facultatively anaerobic bacterium, particularly said hgdA is at least 60%, 65%, 70%, 75%, 80%, 85%, 90%, 95% or >95% identical, with respect to its amino acid sequence, to hgdA of Clostridium symbiosum, more particularly hgdA is at least 60%, 65%, 70%, 75%, 80%, 85%, 90%, 95% or >95% identical to SEQ NO 4 and has a catalytic activity of at least 75% of the activity of SEQ NO 4 and/or
d. the B subunit of the protein hgd is encoded by a gene derived from a strictly or facultatively anaerobic bacterium, particularly said hgdB is at least 60%, 65%, 70%, 75%, 80%, 85%, 90%, 95% or >95% identical, with respect to its amino acid sequence, to hgdB of Clostridium symbiosum, more particularly hgdB is at least 60%, 65%, 70%, 75%, 80%, 85%, 90%, 95% or >95% identical to SEQ NO 5 and has a catalytic activity of at least 75% of the activity of SEQ NO 5 and/or
e. the C subunit of the protein hgd is encoded by a gene derived from a strictly or facultatively anaerobic bacterium, particularly said hgdC is at least 60%, 65%, 70%, 75%, 80%, 85%, 90%, 95% or >95% identical, with respect to its amino acid sequence, to hgdC of Acidaminococcus fermentans, more particularly hgdC is at least 60%, 65%, 70%, 75%, 80%, 85%, 90%, 95% or >95% identical to SEQ NO 6 and has a catalytic activity of at least 75% of the activity of SEQ NO 6 and/or
f. the protein gcdH is encoded by a gene derived from a strictly or facultatively anaerobic bacterium, particularly said gcdH is at least 60%, 65%, 70%, 75%, 80%, 85%, 90%, 95% or >95% identical, with respect to its amino acid sequence, to gcdH of Pseudomonas aeruginosa, more particularly gcdH is at least 60%, 65%, 70%, 75%, 80%, 85%, 90%, 95% or >95% identical to SEQ NO 7 and has a catalytic activity of at least 75% of the activity of SEQ NO 7 and/or
g. the protein ter is encoded by a gene derived from a strictly or facultatively anaerobic bacterium, particularly said ter is at least 60%, 65%, 70%, 75%, 80%, 85%, 90%, 95% or >95% identical, with respect to its amino acid sequence, to ter of Treponema denticola, more particularly ter is at least 60%, 65%, 70%, 75%, 80%, 85%, 90%, 95% or >95% identical to SEQ NO 8 and has a catalytic activity of at least 75% of the activity of SEQ NO 8 and/or
h. the protein adhE1 is encoded by a gene derived from a strictly or facultatively anaerobic bacterium, particularly said adhE1 is at least 60%, 65%, 70%, 75%, 80%, 85%, 90%, 95% or >95% identical, with respect to its amino acid sequence, to adhE1 of Clostridium acetobutylicum, more particularly adhE1 is at least 60%, 65%, 70%, 75%, 80%, 85%, 90%, 95% or >95% identical to SEQ NO 9 and has a catalytic activity of at least 75% of the activity of SEQ NO 9 and/or
i. the protein adhE2 is encoded by a gene derived from a strictly or facultatively anaerobic bacterium, particularly said adhE2 is at least 60%, 65%, 70%, 75%, 80%, 85%, 90%, 95% or >95% identical, with respect to its amino acid sequence, to adhE2 of Clostridium acetobutylicum, more particularly adhE2 is at least 60%, 65%, 70%, 75%, 80%, 85%, 90%, 95% or >95% identical to SEQ NO 13 and has a catalytic activity of at least 75% of the activity of SEQ NO 13.

7. The method according to claim 1, wherein said transgenic cell comprises one or more plasmids encoding said heterologously expressed enzymes under control of a promoter sequence operable in said cell, particularly a T7 promoter, a lac promoter, a trp promoter, a tac promoter or a λP_{L} promoter.

8. The method according to claim 1, wherein said fermentation step is performed under anaerobic conditions at 25 to 37° C., particularly at 30° C.

9. The method according to claim 1, wherein the medium comprises 8-12 g·L⁻¹ glucose, 8-10 g·L⁻¹ dibasic sodium phosphate dihydrate, 6-8 g·L⁻¹ monobasic potassium phosphate, 0.5-0.7 g·L⁻¹ sodium chloride, 1.2-1.5 g·L⁻¹ magnesium sulphate, 0.03-0.05 g·L⁻¹ calcium chloride dihydrate, 0.8-1.2 g·L⁻¹ ammonium chloride, and 8-12 mmol·L⁻¹ sodium bicarbonate, 0.1-0.15 μg·L⁻¹ selenium, 0.08-0.12 μg·L⁻¹ nickel, 0.7-0.9 μg·L⁻¹ molybdenum, ampicillin, spectinomycin, and kanamycin and neutral pH, particularly pH 6.8-7.3.

10. The method according to claim 7, wherein said plasmid comprises
a. a lac, tac or T7 promoter, and the expression of said heterologous genes is induced by adding IPTG (Isopropyl β-D-1-thiogalactopyranosid) to the medium, particularly 0.1-1 mmol·L⁻¹ IPTG, more particularly 0.5 mmol·L⁻¹ IPTG;
b. a trp promoter, and the expression of heterologous genes is induced by adding 3-b-indoleacrylic acid to the medium, at concentrations ranging from 10 μg·mL⁻¹ to 100 μg/m·L⁻¹;
c. a λP_{L} promoter, and the expression of heterologous genes is induced by increasing the temperature to 42° C.

11. A transgenic cell, wherein the following enzymes are expressed:
a. 2-hydroxyglutarate dehydrogenase hgdH (EC 1.1.99.2.);
b. glutaconate-CoA transferase gctAB (EC 2.8.3.12);
c. (R)-2-hydroxyglutaryl-CoA dehydratase subunits A, B and C hgdABC (EC 4.2.1.167);
d. glutaryl CoA dehydrogenase gcdH (EC 1.3.8.6.);
e. trans-2-enoyl-CoA reductase (NAD+) ter (EC 1.3.1.44.); and
f. a bifunctional aldehyde/alcohol dehydrogenase (NAD+) selected from adhE1 and adhE2 (EC 1.1.1.11/1.2.1.3.);
wherein at least 4 enzymes are expressed heterologously, particularly 5 or 6 enzymes are expressed heterologously.

12. The cell according to claim 11, wherein the cell is selected from genera Escherichia, Corynebacterium, Ralstonia, Clostridium, Pseudomonas, Lactobacillus, Lactococcus, Acidaminococcus, Fusobacterium, Peptoniphilus, Saccharomyces, Streptomyces Lactobacillus, Pichia, Kluyveromyces, Yarrowia, or Staphylococci, particularly Escherichia coli.

13. The cell according to claim 11, wherein
a. the protein hgdH is encoded by a gene derived from a strictly or facultatively anaerobic bacterium, particularly said hgdH is at least 60%, 65%, 70%, 75%, 80%, 85%, 90%, 95% or >95% identical, with respect to its amino acid sequence, to hgdH of Acidaminococcus fermentans, more particularly hgdH is at least 60%, 65%, 70%, 75%, 80%, 85%, 90%, 95% or >95% identical to SEQ NO 1 and has a catalytic activity of at least 75% of the activity of SEQ NO 1 and/or
b. the protein gctAB is encoded by a gene derived from a strictly or facultatively anaerobic bacterium, particularly said gctAB is at least 60%, 65%, 70%, 75%, 80%, 85%, 90%, 95% or >95% identical, with respect to its amino acid sequence, to gctAB of Acidaminococcus fermentans, more particularly gctAB is at least 60%, 65%, 70%, 75%, 80%, 85%, 90%, 95% or >95% identical to SEQ NO 2 and has a catalytic activity of at least 75% of the activity of SEQ NO 2 and/or subunit B of gctAB is at least 60%, 65%, 70%, 75%, 80%, 85%, 90%, 95% or >95% identical to SEQ NO 3 and has a catalytic activity of at least 75% of the activity of SEQ NO 3 and/or
c. the A subunit of the protein hgd is encoded by a gene derived from a strictly or facultatively anaerobic bacterium, particularly said hgdA is at least 60%, 65%, 70%, 75%, 80%, 85%, 90%, 95% or >95% identical, with respect to its amino acid sequence, to hgdA of Clostridium symbiosum, more particularly hgdA is at least 60%, 65%, 70%, 75%, 80%, 85%, 90%, 95% or >95% identical to SEQ NO 4 and has a catalytic activity of at least 75% of the activity of SEQ NO 4 and/or
d. the B subunit of the protein hgd is encoded by a gene derived from a strictly or facultatively anaerobic bacterium, particularly said hgdB is at least 60%, 65%, 70%, 75%, 80%, 85%, 90%, 95% or >95% identical, with respect to its amino acid sequence, to hgdB of Clostridium symbiosum, more particularly hgdB is at least 60%, 65%, 70%, 75%, 80%, 85%, 90%, 95% or >95% identical to SEQ NO 5 and has a catalytic activity of at least 75% of the activity of SEQ NO 5 and/or
e. the C subunit of the protein hgd is encoded by a gene derived from a strictly or facultatively anaerobic bacterium, particularly said hgdC is at least 60%, 65%, 70%, 75%, 80%, 85%, 90%, 95% or >95% identical, with respect to its amino acid sequence, to hgdC of Acidaminococcus fermentans, more particularly hgdC is at least 60%, 65%, 70%, 75%, 80%, 85%, 90%, 95% or >95% identical to SEQ NO 6 and has a catalytic activity of at least 75% of the activity of SEQ NO 6 and/or
f. the protein gcdH is encoded by a gene derived from a strictly or facultatively anaerobic bacterium, particularly said gcdH is at least 60%, 65%, 70%, 75%, 80%, 85%, 90%, 95% or >95% identical, with respect to its amino acid sequence, to gcdH of Pseudomonas aeruginosa, more particularly gcdH is at least 60%, 65%, 70%, 75%, 80%, 85%, 90%, 95% or >95% identical to SEQ NO 7 and has a catalytic activity of at least 75% of the activity of SEQ NO 7 and/or
g. the protein ter is encoded by a gene derived from a strictly or facultatively anaerobic bacterium, particularly said ter is at least 60%, 65%, 70%, 75%, 80%, 85%, 90%, 95% or >95% identical, with respect to its amino acid sequence, to ter of Treponema denticola, more particularly ter is at least 60%, 65%, 70%, 75%, 80%, 85%, 90%, 95% or >95% identical to SEQ NO 8 and has a catalytic activity of at least 75% of the activity of SEQ NO 8 and/or
h. the protein adhE1 is encoded by a gene derived from a strictly or facultatively anaerobic bacterium, particularly said adhE1 is at least 60%, 65%, 70%, 75%, 80%, 85%, 90%, 95% or >95% identical, with respect to its amino acid sequence, to adhE1 of Clostridium acetobutylicum, more particularly adhE1 is at least 60%, 65%, 70%, 75%, 80%, 85%, 90%, 95% or >95% identical to SEQ NO 9 and has a catalytic activity of at least 75% of the activity of SEQ NO 9
i. the protein adhE2 is encoded by a gene derived from a strictly or facultatively anaerobic bacterium, particularly said adhE2 is at least 60%, 65%, 70%, 75%, 80%, 85%, 90%, 95% or >95% identical, with respect to its amino acid sequence, to adhE2 of Clostridium acetobutylicum, more particularly adhE2 is at least 60%, 65%, 70%, 75%, 80%, 85%, 90%, 95% or >95% identical to SEQ NO 13 and has a catalytic activity of at least 75% of the activity of SEQ NO 13.

14. The cell according to claim 11, wherein said cell comprises the sequences for said heterologously expressed enzymes under control of a promoter sequence operable in said cell, particularly a T7 promoter, a lac promoter, a trp promoter, a tac promoter or a λP_{L} promoter.


SEQUENCE LISTING (28 sequences)

SEQ ID NO: 1 - protein, 331 aa, Acidaminococcus fermentans
MKVLCYGVRDVELPIFEACNKEFGYDIKCVPDYLNTKETAEMAAGFDAVILRGNCFANKQNLDIYKKLGVKYILTRTAGTDHIDKEYAKELGFPMAFVPRYSPNAIAELAVTQAMMLLRHTAYTTSRTAKKNFKVDAFMFSKEVRNCTVGVVGLGRIGRVAAQIFHGMGATVIGEDVFEIKGIEDYCTQVSLDEVLEKSDIITIHAPYIKENGAVVTRDFLKKMKDGAILVNCARGQLVDTEAVIEAVESGKLGGYGCDVLDGEASVFGKDLEGQKLENPLFEKLVDLYPRVLITPHLGSYTDEAVKNMVEVSYQNLKDLAETGDCPNKIK

SEQ ID NO: 2 - protein, 320 aa, Acidaminococcus fermentans
MSKVMTLKDAIAKYVHSGDHIALGGFTTDRKPYAAVFEILRQGITDLTGLGGAAGGDWDMLIGNGRVKAYINCYTANSGVTNVSRRFRKWFEAGKLTMEDYSQDVIYMMWHAAALGLPFLPVTLMQGSGLTDEWGISKEVRKTLDKVPDDKFKYIDNPFKPGEKVVAVPVPQVDVAIIHAQQASPDGTVRIWGGKFQDVDIAEAAKYTIVTCEEIISDEEIRRDPTKNDIPGMCVDAVVLAPYGAHPSQCYGLYDYDNPFLKVYDKVSKTQEDFDAFCKEWVFDLKDHDEYLNKLGATRLINLKVVPGLGYHIDMTKEDK

SEQ ID NO: 3 - protein, 266 aa, Acidaminococcus fermentans
MADYTNYTNKEMQAVTIAKQIKNGQVVTVGTGLPLIGASVAKRVYAPDCHIIVESGLMDCSPVEVPRSVGDLRFMAHCGCIWPNVRFVGFEINEYLHKANRLIAFIGGAQIDPYGNVNSTSIGDYHHPKTRFTGSGGANGIATYSNTIIMMQHEKRRFMNKIDYVTSPGWIDGPGGRERLGLPGDVGPQLVVTDKGILKFDEKTKRMYLAAYYPTSSPEDVLENTGFDLDVSKAVELEAPDPAVIKLIREEIDPGQAFIQVPTEAK

SEQ ID NO: 4 - protein, 480 aa, Clostridium symbiosum
MAKQVSPGVLALRKVVDDVHKEAREAKARGELVGWSSSKFPCELAAAFDLNVMYPENQAAGIAANRYGEMMCQAAEDLGYDNDICGYARISLAYAAGVRVSRKYDAETGEYIIDPATGKPLKDAEGNVVIDEATGKPKKDPKTQTPYLVLDNLLEIEALPDGPEKERRLEAISPIRQMRIPQPDFVLCCNNICNCMTKWYENIARMCNVPLIMIDIPYNNTVEVHDDNVKYVRAQFDKAIKQLEELTGKKFDEKKFEKACSNANRTAQAWLKVCDYLQYKPAPYSGFDLFNHMADVVTARARVEAAEAFELLADDLEETVKKGETTTPFPEKYRVMFEGIPCWPKLPNLFKPLKEHGVNVTAVVYAPAFGFVYNNIDEMARAYYKAPNSVCIEQGVDWREGICRDNKVDGVLVHYNRSCKPWSGYMAEMQRRFTEDLGVPCAGFDGDQADPRNFNAAQYETRVQGLVEAMEANKQAKEAK

SEQ ID NO: 5 - protein, 383 aa, Clostridium symbiosum
MSINALLDEFKVKAATPKQQLAEYKAQGKKVIGVLPYYAPEELVYAAGMVPMGIWGSNNKTISRAKEYCATFYCTIAQLALEMLLDGTMDQLDGIITPTICDTLRPMSQNFRVAMGDKMAVIFLAQPQNRFEDFGLQFSVDQYTNVKKELEKVAGKEITNEAIQDAIKVYNKSRAARRKFVELASAHCDVITPTKRSAVLKSFFFMEKPEYIEKLEELNAELEKLPVCDWQGTKVVTSGIICDNPKLLEIFEENNIAIAADDVGHESRSFRVDAPEDEADALMALAKQFANMDYDVLLYDPKSTENRRGEFIANMVKESGAQGLVLFMQQFCDPEEMEYPYLKKALNNAGIPHIKLGIDQQMRDFGQASTAIQAFADVLEMQK

SEQ ID NO: 6 - protein, 260 aa, Acidaminococcus fermentans
MSIYTLGIDVGSTASKCIILKDGKEIVAKSLVAVGTGTSGPARSISEVLENAHMKKEDMAFTLATGYGRNSLEGIADKQMSELSCHAMGASFIWPNVHTVIDIGGQDVKVIHVENGTMTNFQMNDKCAAGTGRFLDVMANILEVKVSDLAELGAKSTKRVAISSTCTVFAESEVISQLSKGTDKIDIIAGIHRSVASRVIGLANRVGIVKDVVMTGGVAQNYGVRGALEEGLGVEIKTSPLAQYNGALGAALYAYKKAAK

SEQ ID NO: 7 - protein, 393 aa, Pseudomonas aeruginosa
MATKASFNWEDPLLLDQQLTEEERMVRDSAQQFAQDKLAPRVLEAFRHEQTDPKIFREMGETGLLGATIPEAYGGSGLNYVCYGLIAREVERVDSGYRSMMSVQSSLVMVPIHEFGNEATRQKYLPKLASGEYIGCFGLTEPNHGSDPGSMVTRAKKVDGGYRLSGSKMWITNSPIADVFVVWAKDDEGQIRGFVLEKGWEGLSAPAIHGKVGLRASITGEIVMDNVFVPEENAFPEVRGLRGPFTCLNSARYGISWGALGAAEFCWHTARQYVLDRQQFGRPLAANQLIQKKLADMQTEITLALQGCLRLGRMKDEGTAAVEITSIMKRNSCGKALDIARLARDMLGGNGISDEFGIARHLVNLEVVNTYEGTHDVHALILGRAQTGIQAFF

SEQ ID NO: 8 - protein, 397 aa, Treponema denticola
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 9 - protein, 862 aa, Clostridium acetobutylicum
MKVTTVKELDEKLKVIKEAQKKFSCYSQEMVDEIFRNAAMAAIDARIELAKAAVLETGMGLVEDKVIKNHFAGEYIYNKYKDEKTCGIIERNEPYGITKIAEPIGVVAAIIPVTNPTSTTIFKSLISLKTRNGIFFSPHPRAKKSTILAAKTILDAAVKSGAPENIIGWIDEPSIELTQYLMQKADITLATGGPSLVKSAYSSGKPAIGVGPGNTPVIIDESAHIKMAVSSIILSKTYDNGVICASEQSVIVLKSIYNKVKDEFQERGAYIIKKNELDKVREVIFKDGSVNPKIVGQSAYTIAAMAGIKVPKTTRILIGEVTSLGEEEPFAHEKLSPVLAMYEADNFDDALKKAVTLINLGGLGHTSGIYADEIKARDKIDRFSSAMKTVRTFVNIPTSQGASGDLYNFRIPPSFTLGCGFWGGNSVSENVGPKHLLNIKTVAERRENMLWFRVPHKVYFKFGCLQFALKDLKDLKKKRAFIVTDSDPYNLNYVDSIIKILEHLDIDFKVFNKVGREADLKTIKKATEEMSSFMPDTIIALGGTPEMSSAKLMWVLYEHPEVKFEDLAIKFMDIRKRIYTFPKLGKKAMLVAITTSAGSGSEVTPFALVTDNNTGNKYMLADYEMTPNMAIVDAELMMKMPKGLTAYSGIDALVNSIEAYTSVYASEYTNGLALEAIRLIFKYLPEAYKNGRTNEKAREKMAHASTMAGMASANAFLGLCHSMAIKLSSEHNIPSGIANALLIEEVIKFNAVDNPVKQAPCPQYKYPNTIFRYARIADYIKLGGNTDEEKVDLLINKIHELKKALNIPTSIKDAGVLEENFYSSLDRISELALDDQCTGANPRFPLTSEIKEMYINCFKKQP

SEQ ID NO: 10 - DNA, 6528 bp, Artificial Sequence
  expression vector pCDFDuet_gctAB_hgdH encoding gctAB and hgdH
ggggaattgtgagcggataacaattcccctgtagaaataattttgtttaactttaataaggagatataccatgggcagcagccatcaccatcatcaccacagccaggatccatgagcaaagtcatgaccctgaaagacgcaatcgcaaaatacgtccacagcggcgaccacatcgccctcggcggcttcaccaccgatcgcaaaccatacgccgcggtgttcgaaatcctgcgccaaggcatcaccgatctgactggtttgggcggcgccgccggtggtgattgggacatgctgattggtaacggtcgcgtgaaggcgtatatcaactgctacaccgcgaacagtggcgttaccaacgtgagtcgccgttttcgcaaatggttcgaagcgggtaaactgactatggaagactattcgcaagatgtgatctacatgatgtggcatgcggcagcgttgggtcttccatttttaccggttacgttgatgcagggcagcggcctgactgatgaatggggcattagcaaggaggttcgcaaaactcttgataaggttccggatgacaagttcaaatacattgataatccgttcaaaccgggtgaaaaagttgtggcggttccggttccgcaggtggatgtcgccattattcacgcacagcaggcatcgccggatggtaccgtccgtatctggggtggtaaatttcaggacgttgacattgcggaagcggccaaatatacgattgtgacgtgcgaggagattatctccgatgaggagatccgtcgtgatccgaccaaaaacgatattccgggcatgtgcgtggatgcggttgtgcttgccccatatggtgcgcatccgtcccagtgctatggcctgtatgattacgataacccgtttctgaaagtgtatgataaagtgtcgaaaacccaggaagactttgatgccttttgtaaggaatgggtcttcgacctgaaagatcacgatgaatatcttaataaacttggcgcaacccgtctgattaatttaaaagtggtgccgggcctgggctatcacattgatatgacgaaagaagataaataattaagaaggagatataccatggccgactacaccaactacaccaacaaagaaatgcaggccgtgaccatcgccaaacagatcaaaaacggccaggtggtgactgttggcaccggcctgccgctgatcggtgcgagcgtcgcaaagcgcgtttatgcgccggattgccacattatcgtcgaaagtggcctgatggattgcagtccggtggaagttccgcgctcggtgggtgatttgcgcttcatggcacactgcggctgtatttggccaaatgtccgtttcgtgggctttgagatcaacgaatatctgcataaagccaatcgccttattgcgtttattggtggtgcccagattgacccgtacggcaacgtcaacagcacgtccatcggcgattatcatcacccaaaaacccgtttcaccggcagcggtggcgcaaatggtatcgccacttacagtaatacgatcattatgatgcaacatgaaaaacgccgctttatgaacaaaatcgactatgtgacctcgccgggctggattgatggcccgggtggccgtgaacgtctcggtctgccgggtgatgtgggtccacagttggttgtgaccgataagggtatcctgaaattcgacgaaaaaaccaaacgcatgtatctggcagcctattacccgacgtccagcccagaggacgtgctggaaaacaccggctttgacctggatgtgagcaaagcagtggaactggaagcgccggacccagcagtgatcaaactgattcgcgaagaaatcgatccgggccaagcatttattcaggttccaactgaagccaaataaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccatgaaagtgctgtgctacggcgttcgcgatgtggaacttccgatcttcgaggcatgtaacaaagaatttggttatgatattaaatgcgtcccggattatctgaataccaaagaaacggcagaaatggccgccggctttgacgcagtgattctccgtggcaactgcttcgcaaacaaacagaacctggatatctacaaaaaactgggcgtcaaatatattctgacgcgtaccgcaggcacggaccacatcgacaaagaatacgcgaaagaacttggctttccgatggccttcgtgccacgctacagtccgaacgccatcgccgagctggccgttacccaggcgatgatgctgctccgccataccgcgtatacgacctcccgcaccgcaaaaaagaacttcaaggtggatgcgtttatgttcagtaaagaagtgcgcaattgcaccgtcggtgttgttggcttaggccgtattggccgcgtcgcggcgcagatctttcatggtatgggtgccactgtgatcggcgaagacgttttcgaaatcaaaggcattgaggattactgcactcaagtgagcttggacgaggttctggaaaagagtgatattatcaccatccacgcgccatatattaaagaaaacggtgcagttgtcacccgcgattttctgaaaaagatgaaagacggtgcgattttggtgaactgcgcacgcggtcagcttgttgacactgaggcagttattgaagcggtggaaagcggtaaacttggcggctacggttgtgatgtgttggatggtgaggcgagcgtctttggtaaggatctcgaaggccaaaaactggaaaacccgttattcgagaaacttgtcgatctttatccgcgtgtgctgattacgccgcatctcggctcctatacggacgaggccgtgaaaaatatggtggaagtgagctaccaaaatctcaaagatctggccgaaactggcgattgcccgaacaaaatcaaataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 11 - DNA, 8346 bp, Artificial Sequence
  expression vector pRSFDuet_gcdH_hgdABC encoding hgdABC and gcdH
ggggaattgtgagcggataacaattcccctgtagaaataattttgtttaactttaataaggagatataccatgggcagcagccatcaccatcatcaccacagccaggatccgaattcgagctcatgagtatctataccctgggcatcgatgtgggcagcaccgccagtaaatgcatcattctgaaagacggcaaagaaatcgtggccaaaagtctggtggccgtgggcaccggcaccagtggcccggcccgcagtattagcgaagtgctggaaaacgcccacatgaaaaaagaagatatggccttcaccctggcaacgggctacggtcgcaacagtctggaaggcatcgccgataaacagatgagcgaactgtcctgccacgcaatgggtgcgtccttcatttggccgaacgtgcataccgtcatcgatattggtggccaagatgtgaaagtcattcatgttgaaaacggtacgatgaccaactttcagatgaacgacaaatgcgcggcaggcactggccgctttctggatgttatggcgaacatcctggaagtcaaggttagcgacttagcagaacttggcgcaaaatccacgaaacgcgtcgcaattagcagcacctgcactgtcttcgcagagagcgaggtcatcagccagctcagcaagggcaccgataaaatcgatatcatcgcaggcatccaccgcagtgtggccagccgcgtcatcggcttagcaaatcgcgtgggcatcgtcaaagacgttgtcatgaccggtggcgttgcgcagaactatggtgttcgtggtgcactggaagagggcctcggcgtcgaaattaaaacctccccgctcgcgcagtataatggcgcgctgggtgcagccctgtacgcatataaaaaagcggcaaaataattaagaaggagatatacaatggcaaaacaagtcagcccaggcgtgctggccctgcgcaaagttgtggacgatgtgcacaaggaagcgcgtgaagccaaagcccgtggcgagttggtgggttggagttcgtccaaattcccgtgcgaacttgcggcagcctttgatctgaacgtcatgtacccggaaaatcaggcggcaggtatcgcagcaaatcgttatggcgaaatgatgtgccaggcagcagaagatcttggttatgataatgatatttgtggctatgcacgcattagtctcgcatacgccgccggcgtgcgtgtttcccgcaaatatgacgcagaaaccggtgagtacattatcgatccggcaacgggcaaaccgctgaaagatgcggaaggcaacgtcgtcattgacgaagcaactggcaagccgaaaaaagacccaaaaacgcagaccccgtacttagtcctggacaatctgctggaaatcgaagcactgccggacggcccagaaaaagaacgccgtctggaagcaatcagcccaattcgccagatgcgcatcccacagccagactttgtcctctgttgcaacaacatctgtaattgcatgacgaaatggtatgaaaacatcgcacgcatgtgcaacgtcccactcatcatgatcgacatcccatacaacaacaccgtcgaagtccatgacgataacgtgaaatacgtgcgcgcgcaatttgacaaagcaatcaaacagctggaagaactgaccggcaaaaaattcgacgaaaaaaaatttgaaaaagcctgtagtaacgcgaaccgcactgcgcaggcctggctcaaagtctgcgactacctccaatacaaaccagcaccgtactcgggcttcgatcttttcaaccatatggccgacgtcgtgaccgcgcgcgcgcgcgtcgaagcggcagaagcgtttgaactgctggccgacgacctcgaggaaaccgtcaaaaagggcgaaaccaccaccccgttcccagaaaaataccgcgtcatgttcgaaggcattccatgctggccgaaactgccaaacttattcaaaccactcaaagaacatggcgtgaacgttaccgcagtcgtgtacgccccagcgttcggcttcgtttataacaatatcgacgaaatggcacgcgcctactacaaagccccaaacagcgtgtgcatcgaacaaggcgtggattggcgcgagggtatctgccgcgacaacaaagtcgatggcgtgctggttcactacaaccgtagctgcaaaccgtggagcggctatatggcagagatgcaacgccgcttcaccgaggatcttggcgttccatgcgccggcttcgatggtgatcaagccgacccgcgcaactttaatgcagcccaatacgaaacccgcgttcaaggccttgtggaggcaatggaagccaacaagcaagcgaaggaagcaaagtaattaagaaggagatataccatgagcatcaacgcactgttggacgaatttaaagtgaaagcggccacgccgaaacaacaacttgcggagtataaagcgcagggtaaaaaggttatcggcgtcctgccatactacgcgccagaagaattggtgtatgcggccggcatggtgccgatgggcatctggggctccaataataaaaccatctcccgtgcgaaagagtactgcgcgaccttctattgcacgatcgcccaattggcactggagatgctgctggatggcacgatggaccagctggacggcattatcaccccgacgatttgcgacaccttgcgcccaatgagccagaactttcgcgtggcgatgggcgataagatggcggtcattttcttggcgcaaccgcagaatcgtttcgaagacttcggcctccaatttagcgttgatcagtataccaacgtcaagaaagaactggaaaaagtggccggtaaagaaattaccaacgaggcgattcaagatgccattaaagtttacaacaagagtcgcgccgcacgtcgtaagttcgttgagttggcgtccgcccattgcgatgtgattaccccgaccaaacgcagcgcggtgttgaaatccttttttttcatggaaaagccggaatacattgagaagctggaggaactcaacgcagaactggaaaaattaccagtgtgtgattggcagggcaccaaagtggtcaccagcggcatcatttgcgataacccaaaactgcttgagatttttgaagagaacaacattgcaattgcggcggatgatgtgggccacgagagtcgcagcttccgtgtggacgcaccagaggatgaggccgatgcccttatggcgttggccaagcaattcgcaaacatggattatgatgttttgctgtatgatccgaaaagcaccgaaaatcgtcgcggcgagttcatcgccaacatggtgaaagagagcggcgcccagggtcttgtgttgtttatgcagcagttctgcgatccagaagaaatggagtacccgtatctgaaaaaggccctgaacaacgcaggcattccgcacattaagttgggcattgatcagcagatgcgtgattttggtcaggcgagtactgcaatccaggcgtttgccgacgttttggagatgcaaaaataagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcaaccaaagcaagcttcaactgggaagacccactgctcttggaccaacaactgaccgaagaggaacgcatggtccgcgatagcgcgcagcaattcgcccaggacaaactggccccacgtgtccttgaagcgtttcgccacgaacagactgatccgaagatcttccgcgaaatgggcgaaaccggcctgctgggtgccaccatcccggaagcctatggcggctcgggtctgaactatgtttgctacggtttgattgcacgtgaggtggaacgtgtggatagcggctatcgtagtatgatgtccgtgcagtccagtctggtgatggtgccgatccacgaatttggcaatgaggcgacgcgccagaaatacctgccgaaattagccagcggtgagtatatcggctgctttggcttaacggaaccgaaccatggttcggatccgggtagcatggtgacgcgtgccaaaaaagttgatggcggttaccgcctgagtggctcgaaaatgtggattaccaactccccaattgcggacgtgtttgtggtttgggcgaaagatgacgagggccaaattcgcggctttgttttggaaaagggttgggaaggtctgagcgccccggcgatccacggcaaagtgggcctccgtgcctcgattactggtgaaatcgttatggataatgtgttcgtcccagaggagaatgcatttccggaagtccgtggtctgcgcggtccgttcacctgtttaaatagtgcacgttacggtattagctggggcgcgttaggcgcggcagaattttgctggcataccgcccgccagtacgttctcgatcgccaacagtttggccgcccgctggcggcgaaccagttaattcagaaaaagttagcggatatgcaaactgagattacgttagccctgcagggctgcctgcgtctgggtcgtatgaaggacgagggtaccgcagcagttgagatcactagcattatgaaacgcaattcgtgtggcaaggcactggatatcgcacgcctggcgcgtgatatgcttggtggtaacggcatctcggacgaatttggtatcgcgcgtcatttagtcaacctggaagtggttaatacctatgaaggtacccatgacgttcatgcactgattttaggtcgtgcccagaccggtattcaagcgttcttttgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 12 - DNA, 9118 bp, Artificial Sequence
  expression vector pETDuet_adhE_ter encoding adhE1 and ter
ggggaattgtgagcggataacaattcccctctagaaataattttgtttaactttaagaaggagatataccatgggcagcagccatcaccatcatcaccacagccaggatccgaattcatgaaagtcacaacagtaaaggaattagatgaaaaactcaaggtaattaaagaagctcaaaaaaaattctcttgttactcgcaagaaatggttgatgaaatctttagaaatgcagcaatggcagcaatcgacgcaaggatagagctagcaaaagcagctgttttggaaaccggtatgggcttagttgaagacaaggttataaaaaatcattttgcaggcgaatacatctataacaaatataaggatgaaaaaacctgcggtataattgaacgaaatgaaccctacggaattacaaaaatagcagaacctataggagttgtagctgctataatccctgtaacaaaccccacatcaacaacaatatttaaatccttaatatcccttaaaactagaaatggaattttcttttcgcctcacccaagggcaaaaaaatccacaatactagcagctaaaacaatacttgatgcagccgttaagagtggtgccccggaaaatataataggttggatagatgaaccttcaattgaactaactcaatatttaatgcaaaaagcagatataacccttgcaactggtggtccctcactagttaaatctgcttattcttccggaaaaccagcaataggtgttggtccgggtaacaccccagtaataattgatgaatctgctcatataaaaatggcagtaagttcaattatattatccaaaacctatgataatggtgttatatgtgcttctgaacaatctgtaatagtcttaaaatccatatataacaaggtaaaagatgagttccaagaaagaggagcttatataataaagaaaaacgaattggataaagtccgtgaagtgatttttaaagatggatccgtaaaccctaaaatagtcggacagtcagcttatactatagcagctatggctggcataaaagtacctaaaaccacaagaatattaataggagaagttacctccttaggtgaagaagaaccttttgcccacgaaaaactatctcctgttttggctatgtatgaggctgacaattttgatgatgctttaaaaaaagcagtaactctaataaacttaggaggcctcggccatacctcaggaatatatgcagatgaaataaaagcacgagataaaatagatagatttagtagtgccatgaaaaccgtaagaacctttgtaaatatcccaacctcacaaggtgcaagtggagatctatataattttagaataccaccttctttcacgcttggctgcggattttggggaggaaattctgtttccgagaatgttggtccaaaacatcttttgaatattaaaaccgtagctgaaaggagagaaaacatgctttggtttagagttccacataaagtatattttaagttcggttgtcttcaatttgctttaaaagatttaaaagatctaaagaaaaaaagagcctttatagttactgatagtgacccctataatttaaactatgttgattcaataataaaaatacttgagcacctagatattgattttaaagtatttaataaggttggaagagaagctgatcttaaaaccataaaaaaagcaactgaagaaatgtcctcctttatgccagacactataatagctttaggtggtacccctgaaatgagctctgcaaagctaatgtgggtactatatgaacatccagaagtaaaatttgaagatcttgcaataaaatttatggacataagaaagagaatatatactttcccaaaactcggtaaaaaggctatgttagttgcaattacaacttctgctggttccggttctgaggttactccttttgctttagtaactgacaataacactggaaataagtacatgttagcagattatgaaatgacaccaaatatggcaattgtagatgcagaacttatgatgaaaatgccaaagggattaaccgcttattcaggtatagatgcactagtaaatagtatagaagcatacacatccgtatatgcttcagaatacacaaacggactagcactagaggcaatacgattaatatttaaatatttgcctgaggcttacaaaaacggaagaaccaatgaaaaagcaagagagaaaatggctcacgcttcaactatggcaggtatggcatccgctaatgcatttctaggtctatgtcattccatggcaataaaattaagttcagaacacaatattcctagtggcattgccaatgcattactaatagaagaagtaataaaatttaacgcagttgataatcctgtaaaacaagccccttgcccacaatataagtatccaaacaccatatttagatatgctcgaattgcagattatataaagcttggaggaaatactgatgaggaaaaggtagatctcttaattaacaaaatacatgaactaaaaaaagctttaaatataccaacttcaataaaggatgcaggtgttttggaggaaaacttctattcctcccttgatagaatatctgaacttgcactagatgatcaatgcacaggcgctaatcctagatttcctcttacaagtgagataaaagaaatgtatataaattgttttaaaaaacaaccttaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgattgtaaaaccaatggttaggaacaatatttgtctaaacgctcatccgcaaggatgcaaaaaaggcgttgaggatcaaatagagtacacaaaaaagagaattaccgctgaggtaaaagccggagcaaaagcccctaaaaacgtgctggttctcggctgctcgaacggttacggacttgcaagccggataacggcagcattcggctatggggccgccactatcggcgtttcctttgaaaaagccggaagcgaaacaaagtacggcacacccggctggtacaacaacctggcctttgacgaggctgccaaaagggaaggcctttattccgtaactatagacggagacgccttttccgatgaaatcaaggcacaagtaatcgaagaagccaaaaagaaaggaattaaattcgatcttatagtttacagcttggcaagccctgtaagaaccgatcccgacacaggcataatgcacaagtccgtcttaaaacccttcggtaaaacatttacaggcaagacagtcgatccctttacgggagaactaaaagaaatctccgccgaacctgcaaacgatgaagaagccgctgcaaccgttaaggttatgggaggagaagactgggaacgctggataaagcagctttcaaaagaaggtcttttagaagaaggctgcattaccctagcctattcctatatcggccctgaggccactcaagccctctaccgaaagggcaccataggaaaggcaaaagaacaccttgaagcaactgcccaccgcctaaacaaagaaaacccgtcaatacgggccttcgtttcggtgaacaagggcttggtaacaagggcaagtgcggtaatccccgtaattcccctatacctcgcttccttgtttaaggttatgaaagaaaaaggaaaccacgagggctgtatcgagcagattacccgcctttatgccgaaagactctaccgtaaagacggcaccatccccgtcgatgaagaaaacagaatccgtatcgacgactgggagcttgaagaagacgttcaaaaggcggtttcggctttaatggaaaaagtaaccggcgaaaatgccgaaagcctaaccgaccttgcaggctaccgccacgactttttagcctcaaacggctttgatgtagaaggcatcaactacgaagccgaggtagaaaggttcgacaggatttaactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 13 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 14 - DNA, 2577 bp, Clostridium acetobutylicum
atgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaa

SEQ ID NO: 15 - DNA, 2577 bp, Artificial Sequence
  codon-optimized gene sequence
atgaaagtgaccaatcagaaagaactgaaacagaaactgaatgaactgcgcgaagcccagaaaaaatttgcaacctatacccaggaacaggtggataaaatttttaaacagtgtgcaattgcagcagccaaagaacgtattaatctggccaaactggccgtggaagaaaccggcattggcttagtggaagataaaattattaaaaatcattttgccgccgaatatatttataataaatataaaaatgaaaaaacctgtggcattattgatcatgatgattctttaggcattaccaaagtggccgaaccgattggtattgttgcagccattgttccgaccaccaatccgacctctaccgccatttttaaatctttaatttcactgaaaacccgtaatgccattttttttagcccgcatccgcgtgcaaaaaaatcaaccattgcagcagccaaactgattctggatgcagccgttaaagcaggcgccccgaaaaatattattggttggattgatgaaccgtcaattgaactgtcacaggatctgatgagcgaagcagatattattctggccaccggcggtccgtctatggttaaagcagcctatagtagcggcaaaccggccattggtgtgggtgcaggtaataccccggccattattgatgaatcagccgatattgatatggccgtgagtagtattattctgtctaaaacctatgataatggtgtgatttgtgcctcagaacagtcaattctggttatgaatagtatttatgaaaaagttaaagaagaatttgttaaacgcggctcttatattctgaatcagaatgaaattgccaaaattaaagaaaccatgtttaaaaatggtgccattaatgcagatattgtgggcaaatcagcctatattattgccaaaatggccggcattgaagttccgcagaccaccaaaattctgattggcgaagttcagtcagtggaaaaatcagaactgttttcacatgaaaaactgtctccggtgttagccatgtataaagttaaagattttgatgaagccctgaaaaaagcacagcgtctgattgaactgggcggtagcggtcatacctcttcactgtatattgattcacagaataataaagataaagttaaagaatttggcttagccatgaaaacctctcgtacctttattaatatgccgtcttcacagggcgcctcaggcgatctgtataattttgccattgccccgagctttaccttaggttgtggcacttggggcggtaatagtgtgagtcagaatgtggaaccgaaacatctgctgaatattaaatcagttgcagaacgtcgcgaaaatatgctgtggtttaaagttccgcagaaaatttattttaaatatggttgtctgcgctttgccctgaaagaactgaaagatatgaataaaaaacgcgcctttattgtgaccgataaggacctgtttaaactgggctatgttaataaaattaccaaagtgttagatgaaattgatattaaatatagcatttttaccgatattaaatcagatccgaccattgatagcgttaaaaaaggcgccaaagaaatgctgaattttgaaccggataccattatttcaattggcggcggtagtccgatggatgcagccaaagtgatgcatctgctgtatgaatatccggaagccgaaattgaaaatctggcaattaattttatggatattcgcaaacgcatttgtaattttccgaaactgggcaccaaagcaattagcgttgccattccgaccaccgcaggcaccggctcagaagcaaccccgtttgccgtgattaccaatgatgaaacaggaatgaaatatccgctgaccagctatgaactgaccccgaatatggccattattgataccgaactgatgctgaatatgccgcgtaaactgaccgccgcaaccggcattgatgccttagttcatgcaattgaagcctatgtgagcgttatggccaccgattataccgatgaactggccctgcgtgcaattaaaatgatttttaaatatctgccgcgcgcctataaaaatgggacgaatgatattgaagcacgcgaaaaaatggcacatgcctctaatattgcaggtatggcctttgccaatgcctttctgggtgtttgtcatagtatggcacataaactgggtgcaatgcatcatgttccgcatggtattgcatgtgccgtgctgattgaagaagtgattaaatataatgcaaccgattgtccgaccaaacagaccgcctttccgcagtataaatctccgaatgccaaacgcaaatatgcagaaattgccgaatatctgaatctgaaaggcaccagcgataccgaaaaagtgaccgccctgattgaagcaatttctaaactgaaaattgatctgtctattccgcagaatatttcagcagcaggcattaataaaaaagatttttataataccttagataaaatgagcgaactggcctttgatgatcagtgtaccaccgccaatccgcgctatccgctgattagcgaactgaaagatatttatattaaatctttttaa

SEQ ID NO: 16 - DNA, 1194 bp, Artificial Sequence
  condon-optimized gene sequence
atgattgttaaaccgatggtgcgtaataatatttgtctgaatgcacatccgcagggctgtaaaaaaggcgtggaagatcagattgaatataccaaaaaacgtattaccgccgaagttaaagcaggtgccaaagccccgaaaaatgtgttagtgctgggttgttctaatggctatggcctggccagtcgcattaccgccgcctttggctatggtgcagcaaccattggtgtgagttttgaaaaagcaggctcagaaaccaaatatggcacccccggttggtataataatttagcctttgatgaagccgccaaacgcgaaggcttatatagcgtgaccattgatggcgatgccttttcagatgaaattaaagcacaggttattgaagaagccaaaaaaaaaggtattaaatttgatctgattgtgtatagcttagcctctccggttcgcaccgatccggataccggcattatgcataaatcagtgctgaaaccgtttggcaaaacctttaccggcaaaaccgttgacccgtttaccggcgaactgaaagaaatttctgccgaaccggccaatgatgaagaagcagcagccaccgttaaagttatgggcggcgaagattgggaacgctggattaaacagctgtctaaagaaggtctgctggaagaaggttgtattaccttagcctatagctatattggtccggaagccacccaggccctgtatcgcaaagggactattggtaaagccaaagaacatctggaagcaaccgcacatcgtctgaataaagaaaatccgtcaattcgcgcctttgtgagcgttaataaaggcttagtgacccgtgccagcgccgttattccggtgattccgctgtatctggcctcactgtttaaagttatgaaagaaaaaggcaatcatgaaggttgtattgaacagattacccgcttatatgccgaacgtctgtatcgtaaagatgggaccattccggtggatgaagaaaatcgtattcgcattgatgattgggaactggaagaagatgttcagaaagcagtgagcgccctgatggaaaaagtgaccggcgaaaatgcagaatcactgaccgatttagcaggctatcgtcatgattttctggcaagtaatggttttgatgtggaaggcattaattatgaagccgaagtggaacgctttgatcgcatttaa

SEQ ID NO: 17 - DNA, 30 bp, Artificial Sequence
  Primer adhE_fw
ccgaattcatgaaagtcacaacagtaaagg

SEQ ID NO: 18 - DNA, 34 bp, Artificial Sequence
  adhE_rev
ccgcggccgcttaaggttgttttttaaaacaatt

SEQ ID NO: 19 - DNA, 19 bp, Artificial Sequence
  Primer ter_fw
cccatatgattgtaaaacc

SEQ ID NO: 20 - DNA, 15 bp, Artificial Sequence
  Primer ter_rev
ccctcgagttaaatc

SEQ ID NO: 21 - DNA, 29 bp, Artificial Sequence
  Primer hgdABC_fw
ccgagctcatgagtatctataccctgggc

SEQ ID NO: 22 - DNA, 31 bp, Artificial Sequence
  Primer hgdABC_rev
ccgcggccgcttatttttgcatctccaaaac

SEQ ID NO: 23 - DNA, 22 bp, Artificial Sequence
  Primer gcdH_fw
cccatatggcaaccaaagcaag

SEQ ID NO: 24 - DNA, 29 bp, Artificial Sequence
  Primer gcdH_rev
ccctcgagtcaaaagaacgcttgaatacc

SEQ ID NO: 25 - DNA, 28 bp, Artificial Sequence
  Primer hgdH_fw
ccggtaccatgaaagtgctgtgctacgg

SEQ ID NO: 26 - DNA, 28 bp, Artificial Sequence
  Primer hgdH_rev
ccctcgagttatttgattttgttcgggc

SEQ ID NO: 27 - DNA, 27 bp, Artificial Sequence
  Primer gctAB_fw
ccggatccatgagcaaagtcatgaccc

SEQ ID NO: 28 - DNA, 29 bp, Artificial Sequence
  Primer gctAB_rev
ccaagcttttatttggcttcagttggaac